(19) United States
(12) Patent Application Publication
(10) Pub. No.: US 2005/0246954 A1
(43) Pub. Date: Nov. 10, 2005

(54) Devices and methods for growing plants

(75) Inventors: W. Michael Bissonnette, Boulder, CO (US); Robert E. Wainwright, Longmont, CO (US); John Thompson, Boulder, CO (US); Carson Payne, Niwot, CO (US); Sylvia Bernstein, Boulder, CO (US); Curt Morgan, Huntington Beach, CA (US); Robert Bromley, Littleton, CO (US); Andrew R. Bridgeman, Longmont, CO (US); Laura Conley, Boulder, CO (US); Ann Forsthoefel, Lafayette, CO (US); Robert Showalter, Denver, CO (US); Frederic Wiedemann, Longmont, CO (US)

Correspondence Address: AeroGrow International, Inc.;Suite 201, 900 28th St., Boulder, CO, 80303, US

(73) Assignee: AeroGrow International, Inc., Boulder, CO (US)

(21) Appl. No.: 11/098,176

(22) Filed: Apr. 4, 2005

Related U.S. Application Data
(62) Division of application No. 10/528,110.
Application No. 10/528,110 is a continuation-in-part of application No. 10/714,786, filed on Nov. 17, 2003.
(60) Provisional application No. 60/553,620, filed on Mar. 16, 2004.
(60) Provisional application No. 60/563,951, filed on Apr. 21, 2004.

(30) Foreign Application Priority Data
Sep. 15, 2004 (WO) PCT/US04/30168

(51) Int. Cl. A01G 31/02

(52) U.S. Cl. 47/62 A

(57) ABSTRACT

This invention provides methods, devices, and kits for growing a plant or germinating a seed into a plant; methods and devices for delivering oxygen to a plant or a seed which will germinate into a plant; methods and devices for delivering liquid to a plant; methods and devices for increasing the dissolved oxygen concentration in a liquid to be delivered to a plant; methods and devices for increasing the dissolved oxygen concentration in a liquid within a hydroponics device; terraced oxygenators; aspirators, downdraft venturis, net baskets; germination caps, sets of germination caps; methods for increasing the likelihood of germination of a seed; seed-bearing support media; methods for germinating a seed; and smart garden devices for hydroponics growing systems.

### FIELD OF THE INVENTION

This invention is in the fields of plant agriculture, home gardening, indoor gardening, and hydroponics.

### BACKGROUND

Hydroponics is the cultivation of plants without soil. Hydroponics provides healthier, disease-free plants, faster than growing in soil. In soil-less culture, plants are instead cultivated using a liquid solution of water and nutrients. There are 6 basic types of hydroponic systems: Wick, Raft (also called Water Culture), Ebb and Flow (also called Flood & Drain), Drip, Nutrient Film Technique, and Aeroponic. There are hundreds of variations on these basic types of systems, and most hydroponics systems can be described as a variation or combination of these six types.

Wick systems can be simple, passive systems, with no moving parts. Plants are grown in a soil-less growing medium and a solution containing water and nutrients is delivered using wicks that absorb the solution from a reservoir and deliver the solution to the growing medium. The roots of the plants are optionally prevented from or allowed to grow in the solution. Plant growth is limited by the delivery rate of the wicks and the amount of oxygen in the solution, which, unless supplemented, is often low.

Raft systems can also be very simple. Plants are grown in a soil-less growth medium that is floated by a raft on the surface of a solution containing water and nutrients. The roots of the plants are optionally prevented from or allowed to grow in the solution. Plant growth is limited by the amount of oxygen in the solution, which, unless supplemented, is often low.

Ebb and Flow systems are more complex. The plants are grown in a soil-less growth medium in a flooding tray. Solution containing water and nutrients is intermittently delivered to the flooding tray and then returned to a reservoir. The plant roots are directly or indirectly contacted by the solution in the flooding tray. Optionally the solution is delivered by a pump and returned by gravity. The flooding cycle is optionally controlled by a timer.

Drip systems are divided into recovery and non-recovery systems. Plants are grown in a soil-less growing medium. A solution containing water and nutrients is delivered in drips to the growing medium. The solution that is not used by the plants is either recycled (recovery systems) or discarded (non-recovery systems). In recovery systems, although there often is a reservoir, the plant roots are typically prevented from growing directly in the solution. Plant growth is limited by the amount of oxygen in the solution, which, unless supplemented, is often low.

Nutrient Film Technique (N.F.T.) systems constantly deliver a thin film of a nutrient and water containing solution. The plants are grown in a soil-less growth medium and the roots are allowed to grow outside the medium into the surrounding air or the plants are grown directly suspended in the air without a growing medium. The roots that grow in the air are constantly contacted by the thin film of solution. Typically the solution is recycled. Optionally the solution is delivered by a pump and returned by gravity. Because there is only a thin film of solution, the roots are very susceptible to drying out if the flow of nutrient solution is interrupted.

Aeroponic systems deliver the solution as a fine spray. The plants are grown in a soil-less growth medium and the roots are allowed to grow outside the medium into the surrounding air or the plants are grown directly suspended in the air without a growing medium. The roots that grow in the air are intermittently sprayed or misted with a solution containing water and nutrients. The roots of the plants are optionally prevented from or allowed to grow in the solution. Typically a timer is used to regulate the spraying cycle. Aeroponic systems often suffer from roots growing into and clogging the sprayers and from large roots close to the sprayer preventing roots further away from being sprayed, both requiring extensive maintenance or resulting in losses of plants. EP 0 052 264, filed Oct. 26, 1981, by Ein-Gedi, is an example of an Aeroponic system.

Aeroponics systems do not employ a means for supporting the roots in a liquid, or in a porous or particulate medium. In an aeroponic system, plants are supported over a chamber. The foliage of the plant extends upward from the outer surface of the chamber where it may be exposed to light and the roots extend downward into the chamber where they are suspended freely and are periodically exposed to a spray, forced mist, fog or other method of nutrient solution delivery. In an aeroponic system, nutrient delivery to the root structure of a plant is even more carefully regulated than in a hydroponic system.

U.S. Pat. No. 5,201,141, issued Apr. 13, 1993, describes a hydroponics system made from a pair of flatwise juxtaposed layers of water-impervious material, to make a system resembling an airless N.F.T. The system is not useful for germination of plant seeds; plants already having roots are inserted. Because the layers are flatwise, there is no distinct airspace in which roots are allowed to grow, and no liquid reservoir in which roots can grow is provided. No drops descend through air. This system does not allow growing medium to be used.

U.S. Pat. No. 5,440,836, issued Aug. 15, 1995, describes a multistorey, stacked bed hydroponics system. No liquid solution is delivered to a reservoir without first contacting a growing medium, plant, or side wall of the reservoir. No drops descend through air.

Neither of the two previously mentioned hydroponics systems allow liquid drops to descend through a gas.

EzHydroKit (EzHydroKit, Tucson, Ariz.) is a drip system that uses rock wool as a growing medium. The rock wool is held in a net pot and micro tubing pumps solution to the net pot where it is sprayed into the net pot. The solution then returns to the reservoir, which must be kept at a level just below the net pots. Keeping the solution at a level just below the net pots prevents the formation of an air space. No liquid solution is delivered to a reservoir without first contacting a growing medium. The method for using the kit as described in their manual (EzGrowGuide™ 2003) requires that the rock wool be soaked overnight at pH 5.5 or less and requires the use of unfiltered water. The manual instructs that the drip system should not be used during the first two weeks of growth, including during germination. The solution is to be changed every 7-10 days, including the method step of pH balancing the water to pH 5.5. The manual instructs that the pump is never to be stopped except for when changing the solution.

U.S. Pat. No. 4,392,327, issued Jul. 12, 1983, and EP 0 042 697, published Dec. 30, 1981, describe a hydroponics system having upper and lower compartments formed of flexible plastics. This system is not useful for germination; plants are added when they already have formed a root ball. In the non-wicking systems, liquid is delivered above the plant transition region. No liquid is delivered to a reservoir without first contacting a growing medium or a compartment wall.

U.S. Pat. No. 6,088,958, issued Jul. 18, 2000, describes a hydroponic system for growing potatoes using a stolon partition member to prevent lenticel hypertrophy. This system is not useful for any plants other than potatoes and is not useful during germination. Liquid is not delivered to the plant at the height of the transition region or to each plant separately.

Neither of the three previously mentioned hydroponics systems is useful for plant seed germination.

U.S. Pat. No. 4,310,990, issued Jan. 19, 1982, describes a hydroponics system made from interfitting tubular elements. No liquid solution is delivered to a reservoir without first contacting a growing medium, and no amount of solution deeper than a thin film is allowed to be inside the lower channel, therefore roots never grow within a solution reservoir.

U.S. Pat. No. 5,394,647, issued Mar. 7, 1995, describes an aeroponic hydroponics system. A horizontal divider separates the roots from the reservoir, preventing the roots from being immersed in the solution. No liquid solution is delivered to a reservoir without first contacting the divider and possibly also the growing medium and/or the plant roots.

WO 94/13129, published Jun. 23, 1994, describes a stacked hydroponics system, which is divided into three horizontal plant husbandry zones. Several methods for delivering liquid are described, however no liquid drops descend into a liquid reservoir. This system is not useful for germination.

Neither of the three previously mentioned hydroponics systems provides a reservoir for the growth of roots.

None of the previously mentioned hydroponics systems delivers liquid through a gas into a liquid reservoir, without having the liquid first contact a growing medium, a portion of a plant, or a wall of the reservoir vessel. None of the previously mentioned hydroponics systems allows liquid to descend in drops through a gas, delivers liquid directly to a liquid reservoir, and is useful for germination of plant seeds.

Hydroponics systems available in the art have been designed for large-scale agriculture. These systems do not work for the retail consumer because they are expensive, large, unsightly, and/or require extensive maintenance. The consumer also had different goals compared to large-scale agriculture; the consumer's concern for harvest quality greatly outweighs the concern for production quantity. There is a need in the art for devices and methods that allow consumers to grow a large variety of plants, in a large variety of contexts, using a large variety of methods. Consumers have a diverse array of demands. A successful product must accommodate a diversity of aesthetic requirements (e.g., visual, auditory, gustatory) and a wide range of reasons for growing (e.g., alternative plant varieties, alternative horticultural methods). Many individuals have little or no experience growing their own food, yet others have extensive experience gardening. Consumers have access to a diversity of water quality, historically a critical factor for successful hydroponic growing. One characteristic consumers typically share is they have a limited amount of space available for growing food and ornamental plants. There is a need in the art for products that allow consumers to easily grow tasty, nutritious, healthy, and/or beautiful fruits, vegetable, herbs, spices, and flowers from seed through harvest in their own homes, even when they have no previous experience growing plants, yet also provides a superior experience for master gardeners. Previous attempts by others to design such a product have failed due to system expense, complexity or simplicity, aesthetics, flexibility (plants number/variety or horticultural practices), lack of system robustness, and/or amount of prior knowledge or care required by the user. This invention provides devices that fit on a counter underneath standard cabinets, in a modern kitchen.

Plants need light, water, nutrients, oxygen, carbon dioxide, appropriate temperatures, and time in order to grow. This invention provides devices and methods for easily growing a wide variety of plants that are healthier and more nutritious than plants grown in soil. This invention provides a novel hydroponics system that is self-contained, useful for germination through harvest, useful for cuttings, is useful with low technology components, is useful for single plants through agricultural production, and provides more oxygen to the plant roots than other hydroponic systems.

It is known in the art that plants grow faster and healthier in the presence of negative ions. It is known in the art that flowforms oxygenate, revitalize, and rejuvenate water (Flowforms, Practical Hydroponics & Greenhouses, pp 60-61). However, no previously available hydroponics systems have incorporated negative ion generators, and/or flowforms inside a hydroponics device. This invention provides hydroponics devices that incorporate negative ion generators and/or flowforms within. The negative ion generators not only benefit the plants, but also the humans and animals in the vicinity. The flowforms continuously cleanse and oxygenate the recycled liquid, increasing the ranges of lower quality water sources that may be input into the devices of this invention.

A challenge in multiple plant container gardening is the even delivery of inputs to every plant. In hydroponics, the rate and method of liquid delivery is critical. Not enough moisture results in the plants dehydrating and dying. Too much water results in choking, drowning, and death. Containers fail when they hold too much or too little water. US 2003/0167688 (published Sep. 11, 2003) describes a plant root development container that has anti-circling channels and air channels, but none of the channels are for containing or guiding a flowing liquid. Although baskets, hydroponics containers, for containing growth media exist in the art, none direct incoming liquid around a contained plant or growth medium. This invention provides devices for regulating the flow of liquid to the growth medium and to each plant. These devices are particularly useful when initiating the flow of liquid, such as for germination, when the liquid must contact a dry, potentially shrunken, growth medium, to reach a dormant or germinating seed.

A challenge in consumer level hydroponics is incorporating a reliable method for reminding the user to regularly care for the growing plants. This invention provides a reliable method for reminding a user to care for the growing plants.

This invention provides a hydroponics device using a previously unknown liquid delivery system for the delivery of liquid. This invention provides hydroponic devices for oxygenating liquid and optionally for revitalizing and rejuvenating the liquid. This invention provides devices for consistently delivering a selected amount of liquid to the growth medium or plant in a hydroponics device. This invention provides previously unknown combinations of aspirator and venturi devices for oxygenating liquid within a hydroponics device. U.S. Pat. No. 6,120,008 (issued Sep. 19, 2000) describes an oxygenating apparatus, but it works under pressure greater than 1 atm and is not useful inside a hydroponics device.

This invention provides hydroponics devices that provide more oxygen than prior art hydroponics devices, resulting in faster growth, healthier plants, and larger or tastier harvests. The plants grown using hydroponics are more nutritious than plants grown in soil.

The devices of this invention are easy to use, and no plant-growing experience or green thumb is required. The hydroponics devices of this invention are self-contained, providing water, plant nutrients, oxygen, carbon dioxide, and photoradiation, providing everything most plants need to grow. The hydroponics devices of this invention are useful from germination through harvest and through plant senescence or plant death. The devices are useful for growing seedlings for transplantation into another growing system. The devices of this invention are useful for growing plants considered difficult to grow, including orchids and plants considered difficult to germinate, including parsley.

The devices of this invention provide a pleasant, soothing waterfall sound, or optionally are quiet. The devices provide negative ions for better plant health and for better health of the humans and animals in the surroundings.

The methods and devices of this invention are useful for single plants through large-scale agricultural operations. This invention provides devices that are less susceptible than other hydroponics systems to harming plants as a result of electricity failures.

Soil-less cultivation of plants can provide many advantages over traditional soil-based cultivation. In a soil-less medium, delivery of nutrients to plant roots can be regulated more easily in order to optimize plant growth. This is done by precisely controlling the composition of a nutrient solution, and then by controlling precisely the frequency that plant roots are exposed to the nutrient solution. Plants grow faster in a soil-less environment because plant roots are not required to expend the energy to push soil particles, and therefore have more energy available for growing.

In hydroponics techniques, plants are grown in the absence of soil and roots are maintained in a substantially liquid environment or humid environment. Instead of soil, the root mass of the plant is either supported within an essentially homogeneous synthetic or natural medium, which is either porous or particulate, or the root mass is immersed within a liquid, while the foliage of the plant is allowed to extend upward from the root support medium where it is exposed to light. Meanwhile, the root structure is exposed to a nutrient solution which may be either wicked up to the roots by means of a porous wicking medium or circulated by means of a pump irrigation system. Either way, nutrient delivery to the root mass may be carefully regulated.

Soil-less media for growing plants are generally composed of materials that have low water-retention characteristics, allowing liquid nutrient solution to flow readily to plant roots and then to drain away so that roots are not constantly soaked in a liquid that may foster rot or the growth of damaging fungi. Soil-less media may be composed of any number of suitable porous substances such as peat moss, wood bark, cellulose, pumice, plastic or polystyrene pellets, vermiculite or foam, for example.

Various soil-less plant growth media are disclosed in the prior art: For example, Dedolph (U.S. Pat. No. 4,221,749) teaches a quantity of soil mixture particles distributed throughout a body of spongy polymer. Moffet (U.S. Pat. No. 4,803,803) discloses a plant growth media “which comprises small tufts of mineral wool.” Anton (U.S. Pat. No. 5,224,292) discloses a “non-woven mat comprising a layer of hollow synthetic organic fibers.” Hsh (U.S. Pat. No. 5,363,593) discloses a synthetic cultivation medium comprised of scrap textile. Kosinski (U.S. Pat. No. 6,555,219) discloses “a soil substitute” comprised of “biodegradable and non-biodegradable polymer fibers.”

All of these above-mentioned inventions provide a fibrous, filamentous or foam support for seed which allows water to pass through. While these disclosures offer an advantage over germinating seeds in soil alone, none of these references, taken alone or in combination offer the advantages of the present invention.

Seed germination is a particular concern in any soil-less cultivation system. Since the soil-less medium must adequately support the seed, the medium must be composed of a material firm enough to hold a seed, seedling or cutting in place until its root and stem structures can form, and yet it must contain characteristics of porosity and low water-retention so that seeds are not immersed in liquid.

A variety of soil-less, specifically seed-germinating media have been disclosed in the prior art. For example, Jones (U.S. Pat. No. 4,075,785) teaches a “discrete media of finite and substantially definite dimensions and having sufficient mechanical integrity and chemical stability to substantially withstand fracturing and degradation . . . as a seed implanted therein germinates and the resulting plant grows to commercial maturity.” Jones describes one such embodiment of this “discrete media” comprising a “peat pellet encased in perforated plastic.”

Dedolph (U.S. Pat. Nos. 4,221,749 and 4,495,310) teaches a “plant growth supporting rooting medium” comprised of polyurethane foam. This patent has been commercialized in the Chia® sponge and the Rapid Rooter® grow sponge, both of which permit seed germination within the sponge. Nir (U.S. Pat. No. 4,332,105) teaches an “aeroponic plant growth and development medium especially suitable for the development of seeds, seedling or cuttings . . . comprising a support member formed of generally coplanar spaced sheets of screen material.” Alternatively, Nir teaches a “plurality of seed containing dishes” which are perforated to allow “its contents [to be] subjected to a mist.” Fraze (U.S. Pat. No. 4,669,217) teaches “a self-containing nutrient plant propagation medium utiliz(ing) a sterile, low water retention, linear foam plastic” within which a seed may be placed for germination. This medium is placed into the “mounting surface” of a hydroponic system which contains holes sized for the medium. More recently, Ishioka (U.S. Pat. No. 5,934,011) teaches “a seedling culture mat comprising a mat which comprises a fibrous substrate or a water-soluble film or paper.” Otake (U.S. Pat. No. 6,240,674) teaches a porous sheet of foamed cells for raising seedlings on an industrial mass-production scale.

Each of these seed germination media may be used to carry a seed until implantation of the entire seed-bearing medium in either a soil-based or soil-less plant growth system. None of these above described disclosures provides the seed support media of the present invention.

It is known that certain seed types germinate at a higher frequency with light and that others germinate at a higher frequency with darkness. This invention provides germination caps for directing light toward or away from seeds for various germination requirements. Although U.S. Pat. No. 4,198.783 (issued Apr. 22, 1980) describes frosted, convex light absorbing elements to intercept and direct light to plants, the elements do not direct light toward germinating seeds or away from plants or seeds. Also, the shapes of the elements appear to be convex in outer shape to prevent external liquid from being contained, by the element, but the elements do not include optical elements for directing light.

### SUMMARY OF THE INVENTION

This invention provides devices for growing a plant or germinating a seed into a plant, wherein the plant may have one or more roots, the device comprising: a vessel for containing a liquid; a means for removably suspending the plant in a gas above the liquid; a means for elevating a first portion of the liquid above the remaining liquid in the vessel and into the gas wherein the first portion of liquid falls through the gas into the remaining liquid; and a means for contacting a second portion of the liquid with the plant, seed, or a growth medium contacting the plant or seed and allowing the second portion of liquid to return to the remaining liquid; whereby the one or more roots are permitted to grow in the gas and in the remaining liquid. Optionally, the means for contacting the second portion of liquid with the plant, seed, or growth medium comprises delivering the second portion of liquid through a channeled net basket. Optionally, the first portion of liquid falls in drops or streams. The above-mentioned device can also include one or more components selected from the group consisting of: terraced oxygenators; aspirators, downdraft venturis, net baskets; germination caps, sets of germination caps; seed-bearing support media; and smart garden devices.

This invention provides kits for growing a plant or germinating a seed into a plant comprising an abovementioned device and instructions for using the device.

This invention provides methods for growing a plant or germinating a seed into a plant, wherein the plant has at least one root, the method comprising: providing a vessel for containing a liquid; providing a means for removably suspending the plant in a gas above the liquid; providing a conduit in fluid communication with the liquid and the gas; and providing a means for delivering and delivering a first portion and a second portion of the liquid through the conduit whereby the first portion of liquid falls through the gas into the remaining liquid in the vessel, and whereby the second portion of liquid contacts the plant, the seed, or a growth medium contacting the plant or seed, and descends into the remaining liquid; whereby the root of the plant is permitted to grow in the gas and in the remaining liquid.

This invention provides methods for delivering oxygen to a plant or seed which will germinate into a plant, the method comprising: providing a plant with at least one root or a seed which will germinate into a plant having at least one root; providing a liquid capable of having oxygen dissolved therein; providing a gas comprising oxygen gas; providing a means for elevating and elevating a portion of the liquid above the remaining liquid; allowing the portion of liquid to fall through the gas into the remaining liquid whereby oxygen gas dissolves in the portion of liquid or the remaining liquid thereby forming oxygenated liquid; and providing a means for contacting and contacting the plant or seed with the oxygenated liquid.

This invention provides methods for increasing the dissolved oxygen concentration in a liquid within a hydroponics device comprising: providing a hydroponics device comprising: a vessel for containing a liquid; a means for removably suspending one or more of a plant, seed, a growth medium for contacting the plant or seed, and/or a net basket in a gas above the liquid; and a means for elevating a first portion and a second portion of the liquid above the remaining liquid and into the gas whereby the first portion of liquid falls through the gas into the remaining liquid in the vessel, and whereby the second portion of liquid can contact the plant, the seed, or a growth medium contacting the plant or seed, and descends into the remaining liquid; whereby the root of the plant is permitted to grow in the gas and in the remaining liquid; elevating the first portion of liquid above the remaining liquid and into the gas; elevating the second portion of liquid above the remaining liquid and into the gas; allowing the first portion of liquid to fall through the gas and into the remaining liquid; and allowing the second portion of liquid to contact the plant, seed, growth medium, or net basket and descend into the remaining liquid; whereby the dissolved oxygen concentration in the first portion of liquid, in the remaining liquid, or in both is increased.

This invention provides terraced aerators comprising: one or more terraces; a means for suspending the terraced aerator all or partially above a liquid reservoir; and below a plant, seed, or a growth medium suspending the plant or seed; wherein: a liquid descending from the plant or seed or growth medium, through a gas comprising oxygen, to the first terrace; and the liquid descending from the first terrace through a gas comprising oxygen into the liquid reservoir; increases the dissolved oxygen content in the liquid or in the liquid reservoir, or both; and wherein each of the liquid descending steps produces a sound of less than about 57 decibels or wherein each of the liquid descending steps dampens the sound produced compared to the liquid descending to the liquid reservoir without contacting the terraced aerator.

This invention provides methods for increasing the dissolved oxygen concentration in a liquid within a hydroponics device comprising: providing a hydroponics device containing a liquid to be delivered to a plant; a gas comprising oxygen above the liquid; a means for elevating a portion of the liquid in the gas above the remaining liquid; a means for delivering the portion of liquid into the gas; and a terraced aerator suspended in the gas above the liquid; elevating a portion of the liquid above the remaining liquid; delivering the portion of liquid into the gas; and allowing the portion of liquid to descend through the gas onto the terraced aerator and into the remaining portion of liquid; wherein the dissolved oxygen concentration in the liquid is increased.

This invention provides aspirators for increasing the dissolved oxygen concentration in a liquid in a hydroponics system, the aspirator comprising a tube in which the liquid flows, wherein the tube comprises a gas inlet for receiving a gas comprising oxygen, whereby when the liquid flows through the tube the gas enters the tube and mixes with the liquid.

This invention provides downdraft venturi devices for increasing the dissolved oxygen concentration in a liquid in a hydroponics system, the venturi comprising: a tube, the tube having an upper, first cross-sectional area and an area of transition to a lower, second, smaller cross-sectional area, the tube for descent of the liquid; and a gas inlet into the tube at about the area of transition; wherein descent of the portion of liquid through the tube draws a gas comprising oxygen into the gas inlet, whereby the gas mixes with the liquid and increases the dissolved oxygen concentration in the liquid.

This invention provides methods for increasing the dissolved oxygen concentration in a liquid to be delivered to a plant, the method comprising: providing a liquid; providing a downdraft venturi in a gas comprising oxygen; delivering the liquid into the top of the downdraft venturi; and allowing the liquid to descend through the downdraft venturi wherein the gas enters into the downdraft venturi and mixes with the liquid.

This invention provides net baskets for supporting and delivering liquid to a plant, a seed that will germinate into a plant, or a growth medium for contacting the seed or plant, the basket comprising at least one channel having a vertical component for transporting liquid wherein the plant or seed grows and wherein a root of the plant and the liquid are allowed to exit through one or more holes in the net basket. Optionally, the net basket also has at least one channel having a horizontal component for transporting liquid, wherein the channel having a horizontal component is in fluid contact with the channel having a vertical component.

This invention provides methods for delivering liquid to a plant or seed that will germinate into a plant comprising: providing a net basket for supporting the plant or seed, the net basket comprising a liquid inlet and a channel having a vertical component for transporting the liquid; delivering a liquid to the liquid inlet; transporting the liquid through the liquid inlet to the channel having a vertical component; transporting the liquid through the channel having a vertical component; and contacting the plant or seed with the liquid; wherein the plant grows and wherein one or more roots of the plant and the liquid are allowed to exit through one or more holes in the net basket.

This invention provides germination caps for increasing the likelihood of germination of a seed relative to an equivalent context without the cap, the cap comprising: a panel comprising at least a partially converging, diverging, refracting, or polarizing lens; and a means for supporting the panel between a photoradiation source and the seed; wherein the panel is at least partially permeable to photoradiation from the photoradiation source.

This invention provides sets of germination caps for increasing the likelihood of germination of a plurality of seed types relative to an equivalent context without the set of caps comprising two or more germination caps wherein a first germination cap comprises: a first panel comprising at least a partially converging lens; and a means for supporting the first panel between a photoradiation source and the plurality of seed types; and a second germination cap comprising: a second panel comprising at least a partially diverging lens; and a means for supporting the second panel between a photoradiation source and the plurality of seed types; wherein the first and second panels are at least partially permeable to photoradiation from the photoradiation source.

This invention provides methods for increasing the likelihood of germination of a seed comprising: providing a seed; providing a liquid and a means for contacting the seed with the liquid; providing a photoradiation source for delivering photoradiation to the seed; providing a means for converging or diverging the photoradiation towards or away from the seed; contacting the seed with the liquid; and delivering the photoradiation to the seed comprising converging or diverging the photoradiation towards or away from the seed; wherein the likelihood of germination of the seed is increased relative to delivering the photoradiation without converging or diverging the photoradiation.

This invention provides methods for increasing the likelihood of germination of a plurality of seed types, the method comprising: providing a plurality of seed types comprising a first seed and a second seed; providing a liquid and a means for contacting the first and second seeds with the liquid; providing a photoradiation source for delivering photoradiation to the first and second seeds; providing a means for converging or diverging the photoradiation towards or away from each of the first and second seeds; contacting the first and second seeds with the liquid; delivering the photoradiation to the first seed comprising converging the photoradiation towards the first seed; and delivering the photoradiation to the second seed comprising diverging the photoradiation away from the second seed; wherein the likelihood of germination of the seed is increased relative to delivering the photoradiation without converging or diverging the photoradiation.

This invention provides seed-support media comprising: a seed-bearing substrate superposed upon a plant growth medium contained within a modular receptacle.

This invention provides methods for germinating a seed comprising: placing a seed supporting and germinating medium comprising a seed-bearing substrate superposed upon a growth medium contained within a modular receptacle; delivering an aqueous liquid to the seed; and; allowing the seed to germinate.

This invention provides smart garden devices for a hydroponics device, the hydroponics device having at least one characteristic or component, the smart garden device comprising: means for delivering electricity to the smart garden device; at least one timer, and means for determining, receiving, sending, or processing data regarding the status of the component or characteristic of the hydroponics device.

### BRIEF DESCRIPTION OF THE DRAWINGS

FIGS. 1A-D are illustrations showing a perspective view, a front view, a side view, and a back view, respectively, of a device, for growing a plant or germinating a seed into a plant, of this invention.

FIGS. 2A-C are illustrations showing a perspective view, a front view, and a back view, respectively, of a device, for growing a plant or germinating a seed into a plant, of this invention.

FIG. 3 is an illustration showing a front longitudinal cross-section perspective view of a device, for growing a plant or germinating a seed into a plant, of this invention.

FIGS. 4A is an illustration showing a side longitudinal cross-section of the upper portion, including the cover, a germination cap, a seed-support medium, downdraft venturi, pump, and a portion of the cover stand, of the device shown in FIGS. 2A-D. FIGS. 4C-E are illustrations showing horizontal cross-sectional views of the downdraft venturi at various heights. FIG. 4B is an illustration showing a perspective view of the lower potion, including the vessel and nutrient basket, of the device shown in FIGS. 2A-D.

FIG. 5A is an illustration of a top perspective view of the portion of the device shown in FIG. 4A. FIG. 5B is an illustration of a perspective ghost view (dashed lines) of the base, including the smart garden, of the device shown in FIGS. 1A-D.

FIG. 6A is an illustration of a lower perspective view of the portion of the device shown in FIG. 4A. FIG. 6B is a detail illustration of the box in FIG. 6A, showing the downdraft venturi. FIG. 6C is a bottom perspective ghost view of the cover shown in FIG. 6A.

FIGS. 7A-C are illustrations of a perspective view of a terraced aerator with curved, liquid-retaining, alternating terraces; a side view of a terraced aerator having flowform terraces, and a perspective view of an terraced aerator having coaxial flat terraces.

FIG. 8A is an illustration of a bottom perspective view of the device shown in FIGS. 1A-D. FIG. 8B is a bottom perspective view of the artificial photoradiation hood of the device shown in FIG. 8A.

FIGS. 9A-E are illustrations showing a perspective view, a front view, a back view, a side view, and a side view with the arm extended, respectively, of the photoradiation apparatus shown in FIGS. 1A-D.

FIG. 10 is an illustration showing a front view of a smart garden display panel of this invention.

FIGS. 11A-D are illustrations showing a top perspective view, a bottom perspective view, a longitudinal cross-sectional view, and a cut-away top perspective view, respectively, of a net basket of this invention. The section for FIG. 11C is marked in FIG. 11A, and the cut-away of FIG. 11D is at the same section as FIG. 11C.

FIGS. 12A-B are illustrations showing a top perspective views of a seed-support medium of this invention. FIG. 12A shows the net basket shown in FIGS. 11A-D. FIG. 12B shows a label. FIG. 12C shows the seed-support medium shown in FIG. 12B with a germination cap.

FIGS. 13A-C are illustrations showing longitudinal cross-sectional views of seed-support media of this invention.

FIG. 14A is an illustration showing a top perspective view of an alternative hydroponics device of this invention. FIG. 14B is an illustration showing a top view of the lower cover of the device show in FIG. 14A. FIG. 14C is an illustration showing a bottom perspective view of the lower cover of the device show in FIG. 14A. FIG. 14D is an illustration showing a detail cross-sectional view of the tube, valve, and venturi shown in FIG. 14C.

### DETAILED DESCRIPTION OF THE INVENTION

As is used in the art and as used herein, a “vessel” is able to contain a liquid and optionally has a bottom wall and/or one or more side walls. The bottom wall can have vertical as well as horizontal components as in a hemisphere. A side wall has a vertical component. Preferably the vessel is not permeable to photoradiation that would interfere with plant growth or would promote growth of unwanted organisms such as algae.

Vessels of this invention are removably coverable by a cover that has at least one plant opening for removably suspending a plant. Preferably covers are not permeable to photoradiation that would interfere with plant growth or would promote growth of unwanted organisms such as algae. Preferably the devices of this invention are also not permeable to liquids except at the plant opening(s) and any other opening functioning in liquid transfer, such as a liquid fill inlet or outlet. Optionally the cover comprises two or more layers, e.g., an upper and lower cover. When a device of this invention comprises an upper cover and a lower cover, both covers have at least one set of plant openings that are horizontally aligned.

As used in the art and as used herein, “conduit” refers to a form having one or more bottom walls and optionally one or more side walls that is able to route a liquid from one location to one or more other locations. If a conduit is utilized to route a liquid horizontally or downhill, the conduit can comprise a vertically curved bottom wall or a bottom wall and one or more side walls. If a conduit is utilized to route a liquid to a higher elevation, the conduit comprises means for enclosing the liquid, e.g., bottom, side, and top walls, except for inlets and outlets. A tube is a type of conduit.

Conduits deliver liquids to one or more locations through conduit exits. As used herein, “delivering liquid separately to each plant” refers to liquid exiting a conduit through one or more exits wherein after exiting, the liquid is first delivered to only one plant. After being received by the one plant, the liquid can contact other plants. Liquid is not delivered separately to each plant when there are one or more centralized exits for delivery to more than one plant at once. Examples of delivery means that are not separate include use of overhead sprinklers that sprinkle two or more plants at once, flooding more than one plant in one vessel, and spraying more than one plant at a time from a single atomizer or nozzle.

As used herein, “substantially vertically downward” refers to about in the direction of gravity. As used herein, “substantially horizontally” refers to about 90 degrees from or about perpendicular to the direction of gravity on earth. As used herein, “three or more horizontal directions” refers to delivery of liquid from exits that having three or more different angle components on a horizontal plane. As used herein, “all horizontal directions” refers to delivery of liquid to effectively surround a plant on a horizontal plane. As used herein, “horizontal plane” refers to a plane about 90 degrees or about perpendicular to gravity on earth.

As used herein, “falls” refers to moving in the direction of gravity, including but not limited to at an acceleration of about or more than 9.8 m/s², including when the only component of movement is in the direction of gravity, while not contacting a solid object. As used herein, “descends along” refers to moving wherein at least one component of movement is in the direction of gravity, while contacting a solid object. As used herein, “descending” refers to falling and descending along and combinations thereof.

As used in the art and as used herein, “drops” refers to a plurality of water molecules that, when in contact, comprise a three-dimensional volume that is larger than mist and atomized particles. Drop volume can be characterized by considering the diameter of a sphere that could be formed by the volume of the drop. Drops useful in the practice of this invention include drops having diameters greater than about 200 microns, greater than about 350 microns, greater than about 500 microns, greater than about 1000 microns, greater than about 2000 microns, and greater than about 5000 microns. A drop in contact with another drop is also useful in the practice of this invention. As used herein, “stream” refers to more than about three drops in contact with each other, including flowing liquid that is not visibly distinguishable into distinct drops.

As used herein, “increases the dissolved oxygen” refers to increasing the concentration of oxygen dissolved in a liquid or, if the dissolved oxygen is at the maximum concentration, as is known in the art, to maintaining the maximum concentration. As used herein, “increasing the negative ions” refers to increasing the number of electrons that are separated from an atom and optionally attached to a different atom resulting in a negative charge, including an oxygen atom having an extra electron.

As used herein, “oxygenated liquid” refers to liquid having oxygen gas dissolved in it. As used herein, “super-oxygenated liquid” refers to liquid that has had the concentration of dissolved oxygen increased or maintained, if at maximum, as a result of an action on it.

As used herein, “hydroponic” refers to plant growing techniques that do not use soil. As used herein, “transition region” refers to the section of a plant where the shoot or shoot meristem transitions into the root or root meristem. The transition region typically exists just below the upper surface of a plant growth medium. As used herein, “optimal growth” refers to plant growth that is optimized to achieve a selected set of characteristics, e.g., fruit harvest, root harvest, leaf harvest, flower production and/or size, and longevity.

As used in the art and as used herein, “nutrients” refers to atoms and molecules in an available form necessary for plant growth in addition to oxygen, hydrogen, and water including calcium, magnesium, sodium, potassium, nitrogen, phosphorus, sulfur, chlorine, iron, manganese, copper, zinc, boron, and molybdenum. Nutrient formulations and recipes are known in the art (see, for example, Resh H. M (2001) Hydroponic Food Production, Sixth Addition, Woodbridge Press Publishing Company, Santa Barbara, Calif., USA). It is known in the art that a liquid that contacts a plant, e.g., liquid used to supply nutrients to a plant, is preferably within a particular pH range. Optimal pH ranges for a variety of plants are known in the art. As used herein, “photoradiation” refers to wavelengths of light of sufficient quantity and quality that allow a plant to grow, as is known in the art. It is known in the art which quantities and wavelengths of photoradiation are preferred for many plants.

As used herein, “hydrophilic” refers to having an affinity for aqueous liquids. A hydrophilic material is optionally capable of absorbing and/or wicking aqueous liquids. As used herein, “wicking means” refers to a means for wicking a liquid. A wicking means can be a wick comprising a wicking material. As is known in the art, materials differ in the ability to wick, which is described as an absorption coefficient. Different materials are able to wick different quantities of liquids at different rates.

The term “growing a plant” as used in herein refers to the process which takes place when appropriate conditions such as water, photoradiation, gas containing oxygen and carbon dioxide, and nutrients are provided to a plant tissue, whether a seed, a cutting, transplant, bulb, tuber, runner, or a plant having roots, resulting in an increase in the mass of plant tissue. The term “cutting” as used herein refers to plant tissue with or without roots taken from an already existing plant.

The term “germinating a seed into a plant” as used herein refers to the process which takers place when appropriate conditions such as water, photoradiation, gas containing oxygen and carbon dioxide are provided to the seed, resulting in the emergence of a plant embryo from the seed.

The term “removably suspending a plant in a gas” as used herein refers to the positioning of a plant so that the tissues of the plant are contacted by the gas where the plant can be removed from the context. The term “means for elevating liquid” as used herein refers to a method or device which transports the liquid to a position which is higher than its original position. The term “means for contacting liquid with plant, seed or growth medium” as used herein refers to a process or device for allowing liquid to come into physical contact with the plant, seed, or growth medium.

The term “growth medium” as used herein refers to any material which permits the growth of plant material or the germination of a seed to take place.

The term “net basket” as used herein refers to a container which has an opening out of which a plant shoot, stem, or leaf can grow, optionally has an opening out of which a plant root can grow, and which can contain a growth medium.

The term “intermittent delivering” as used herein refers to a delivery schedule which includes periods of time when delivery is not taking place. The term “continuous delivering” as used herein refers to a delivery schedule which does not include a period of time when delivery is not taking place.

The term “downdraft venturi” as used herein refers to a tubular structure having a cross sectional diameter which decreases with decreasing elevation thereby creating a narrowing constriction which results in an increased velocity of a first fluid which travels through the tube. An opening at or near the vicinity of the constriction acts as a portal through which a second fluid is drawn into the tubular structure and the second fluid joins with the first fluid. In an embodiment, the first fluid is a liquid and the second fluid is a gas.

As used herein, “aspirator” refers to a tubular structure in which a first fluid can flow. An opening in the tubular structure acts as a portal through which a second fluid is drawn into the tubular structure and joins with the first fluid. In an embodiment, the first fluid is a liquid and the second fluid is a gas.

The term “terraced aerator” as used herein refers to a device which receives a liquid and allows the liquid to fall in drops or streams a selected distance between terraces. The configuration of the device is selected to optimize oxygenation of the liquid and the level of sound produced. Although the applicant does not want to be held to any theory, it is believed that the contact with a gas comprising oxygen while falling and/or the process of falling on a terrace or into another portion of liquid increases the oxygen concentration in the liquid.

The term “germination cap” as used herein refers to a device which covers a surface of a growing medium. The term “set of germination caps” refers to more than one germination cap.

The term “humidity” as used herein refers to aqueous vapor in a gas. The term “splashing” as used herein refers to the process of allowing second drops or droplets to contact a surface wherein the second drops or droplets form from a liquid as a result of a first drop or stream falling into the liquid.

The term “flowform” as used herein as is known in the art and refers to a contoured surface which directs the flow of a liquid and which, when placed in a gas, is capable of allowing the gas to combine with the flowing liquid.

The term “dissolved oxygen concentration” as used herein refers to the amount of molecular oxygen which is contained in a liquid.

As used herein, the term “open conduit” refers to a conduit which is absent a portion of its outer perimeter.

As used in the art and as used herein, “channel” refers to a form having one or more side walls and optionally one or more bottom walls, wherein the channel is able to route a liquid from a first location to one or more second locations.

Soil-less media for growing plants are generally composed of materials that have moderate water-retention characteristics, allowing liquid nutrient solution to flow readily to plant roots and then to drain away so that roots are not constantly soaked in a liquid that may foster rot or the growth of damaging fungi. Soil-less media may be composed of any number of suitable porous substances such as peat moss, wood bark, cellulose, pumice, plastic or polystyrene pellets, vermiculite or foam, for example.

As used herein, the term “enclosed” refers to the state of having substantially all of the surfaces of a vessel defined by a solid object.

As used herein the term “means for suspending terrace” refers to one or more support structures to which a terrace is attached. As used herein, the term “dampens the sound” refers decreasing in noise level. As used herein, the term “drop distance” refers to the distance a drop of liquid falls from contact with a first solid or liquid to a second solid or liquid.

As used herein, the term “reservoir liquid” refers to the liquid contained in a reservoir.

As used herein, the term “hydrophilic cellular substrate” refers to any material which has an affinity for aqueous liquids, is of a cellular structure, and can function as a growth medium.

As used herein, the term “clog prevention element” refers to any physical device which reduces impediments to the flow of liquid. As used herein, the term “substantially seals” refers to the relationship between one physical element and another physical element wherein no substantial amount of liquid can penetrate. As used herein, the term “distal wall of channel” refers to the outermost portion of the channel wall. As used herein, the term “proximal wall of channel” refers to the innermost portion of the channel wall. As used herein, the term “U-shaped” refers to a geometric configuration which has two sides and a bottom. As used herein the term “L-shaped” refers to a geometric configuration which has one side and a bottom.

As used herein, the term “relative to an equivalent context without said element” refers to a comparative situation in which one instance comprises a described element and the second instance is absent the described element, but is otherwise equivalent.

As used herein, the term “lens” refers to a substance such that at least some photoradiation rays that can pass through it are modified and may or may not be made to change their direction. As used herein, the term “decreasing evaporation of liquid contacting a seed” refers to a condition wherein humidity is substantially prevented from escaping. As used herein, the term “airtight seal” refers to the relationship between one physical element and another physical element wherein substantially no air or gas can penetrate. As used herein, a “panel” is at least a portion of a top, side, or bottom wall.

As used herein, the term “translucent” refers to material which allows some photoradiation rays to pass through, but not so as to render the shape or form of an object on the opposite side of the material distinctly discernible. As used herein, the term “transparent” refers to material which allows enough photoradiation rays to pass through so as to render the shape or form of an object on the opposite side of the material distinctly discernible.

As used herein, “photoradiation includes direct, indirect, reflected, and refracted photoradiation. As used herein, the term “natural or artificial photoradiation source” refers to any source of photoradiation, including the sun, bulbs, and reflective surfaces.

As used herein, the term “greenhouse environment” refers to a set of conditions which approximates the conditions inside a greenhouse. As used herein, the term “terrarium environment” refers to a set of conditions which approximates the conditions inside a terrarium.

As used herein, the term “converging lens” refers to a lens which causes substantially parallel photoradiation rays passing therethrough to converge to a point on the opposite the side of the lens from which the rays came. As used herein, the term “diverging lens” refers to a lens which causes substantially parallel photoradiation rays passing therethrough to diverge, spread out, or trace back to a point on the side of the lens from which the photoradiation came. As used herein, the term “focused photoradiation” refers to photoradiation that has been modified by passage through a lens.

As used herein, the term “characteristic” refers to qualities or attributes which describe the physical condition or state of existence of the device, including, but not limited to: timing cycle, need for nutrients, need for liquid within the device, humidity, root density, nutrient concentration, pH, dissolved oxygen concentration, turbidity of liquid, incident photoradiation, temperature, and plant mass.

As used herein the term “component” refers to physical elements of the device including, but not limited to: timers, photoradiation sources, and pumps.

As used herein, the term “delivering electricity” refers to providing means for allowing electricity to enter and drive the electrical components of the device. The most likely form this electricity delivery will take is to supply a set of wires which can be plugged into household alternating current, but adapting the device for use with a battery operated system is also contemplated.

As used herein, the term “displaying” refers to a visual means of communication of information, such as an illuminated lamp, LCD or liquid level gauge. As used herein, the term “two week cycle” refers to a timing cycle which extends approximately two weeks in duration.

As used herein, the term “liquid nutrient solution” refers to a liquid which contains nutrients in solution or suspension or in a mixture, or in a combination of solution, suspension or mixture. As used herein, the term “nutrient concentration” refers to the concentration of nutrient in the liquid within the device including that which is available for delivery to plant tissue.

As used herein the term “determining, receiving, sending or processing data” refers to one or more operations to a data set which results in the creation of an additional data set. The additional data set can be a copy of the first data set in a new location.

As used herein, the term “programmable storage device” refers to any storage device such as a computer chip, for example, which is capable of storing data and information for executing a program. As used herein the term “preprogrammed storage device” refers to any programmable storage device which is programmed to carry out specific functions.

As used herein, the term “root density” refers to the proportion of root mass in a specific volume, such as g/mm³, for example. As used herein, the term “turbidity” refers to the quantity of suspended material in a liquid, as measured by a photodensitometer.

As used herein, “adjuvants” refers to additives that enhances the effectiveness a composition.

The components illustrated in the drawings are numbered as shown below.

Drawing Elements

| Item | Number |
|---|---|
| 1 | plant growing device |
| 2 | plant |
| 3 | cover |
| 4 | opening |
| 5 | drop falling into liquid |
| 6 | drop falling onto root |
| 7 | root growing in gas |
| 8 | drop falling off root |
| 9 | root growing into liquid |
| 10 | gas |
| 11 | liquid |
| 12 | vessel |
| 13 | pump |
| 14 | conduit |
| 15 | conduit exit |
| 16 | plant support |
| 17 | drop guard |
| 18 | artificial photoradiation source |
| 19 | natural photoradiation source |
| 20 | arrow showing liquid delivery for first portion |
| 21 | tube |
| 22 | drop descending along root |
| 23 | drop falling from plant support |
| 25 | drop height |
| 28 | exit for first portion |
| 29 | exit for second portion |
| 33 | liquid level gauge |
| 41 | liquid directing means |
| 42 | terrace |
| 43 | terrace support means |
| 44 | aerator support means |
| 45 | terrace wall |
| 46 | terrace wall opening |
| 47 | terraced aerator |
| 48 | lower cover leg |
| 100 | hydroponic device with photoradiation apparatus and smart garden |
| 101 | base |
| 102 | door |
| 103 | nutrient inlet cover |
| 104 | means for lifting cover |
| 105 | door hinge |
| 106 | flat bottom surface of device |
| 107 | optional power cord exit |
| 108 | indentation for photoradiation arm |
| 109 | growth medium |
| 110 | seed |
| 111 | adhesive |
| 112 | dibble |
| 113 | pump inlet |
| 114 | leg |
| 115 | nutrient basket |
| 116 | nutrient basket outlet |
| 117 | stand |
| 118 | cover support arm |
| 119 | filter |
| 120 | flowform |
| 121 | means for receiving vessel |
| 122 | growth medium upper surface |
| 220 | artificial photoradiation hood |
| 221 | adjustable photoradiation arm |
| 222 | vent holes |
| 223 | means for adjusting photoradiation hood height |
| 124 | photoradiation source |
| 125 | photoradiation apparatus |
| 126 | extended arm |
| 127 | extension unit height |
| 128 | extension notch |
| 129 | aspirator |
| 130 | downdraft venturi |
| 131 | first cross-sectional area |
| 132 | second, smaller cross-sectional area |
| 133 | third cross-sectional area |
| 134 | venturi exit |
| 135 | gas inlet |
| 140 | smart garden display panel |
| 141 | transformer |
| 142 | circuit board power |
| 143 | circuit board controller |
| 144 | timing cycle selection button for photoradiation source |
| | and/or pump |
| 145 | add nutrients reset button |
| 146 | add nutrients flashing signal |
| 147 | add water flashing signal |
| 148 | timing cycle selection name that lights up |
| 149 | photoradiation cycle override button |
| 150 | germination cap |
| 151 | lens |
| 160 | net basket |
| 161 | channel with a vertical component |
| 162 | channel with a horizontal component |
| 163 | proximal edge of wall of channel having a vertical component |
| 164 | proximal wall of channel having a horizontal component |
| 165 | proximal side of distal wall of channel having a horizontal |
| | component |
| 166 | net basket hole |
| 167 | net basket liquid inlet |
| 168 | basket support means |
| 169 | side of L-shaped horizontal channel |
| 170 | bottom of L-shaped horizontal channel |
| 171 | U-shaped horizontal channel |
| 172 | label |
| 173 | slit in label |
| 180 | seed support medium |
| 182 | rigid modular receptacle |
| 184 | concave recess |
| 185 | seed bearing substrate |
| 186 | support means |
| 187 | seal |
| 188 | valve |
| 189 | optional proximal wall cross-section |

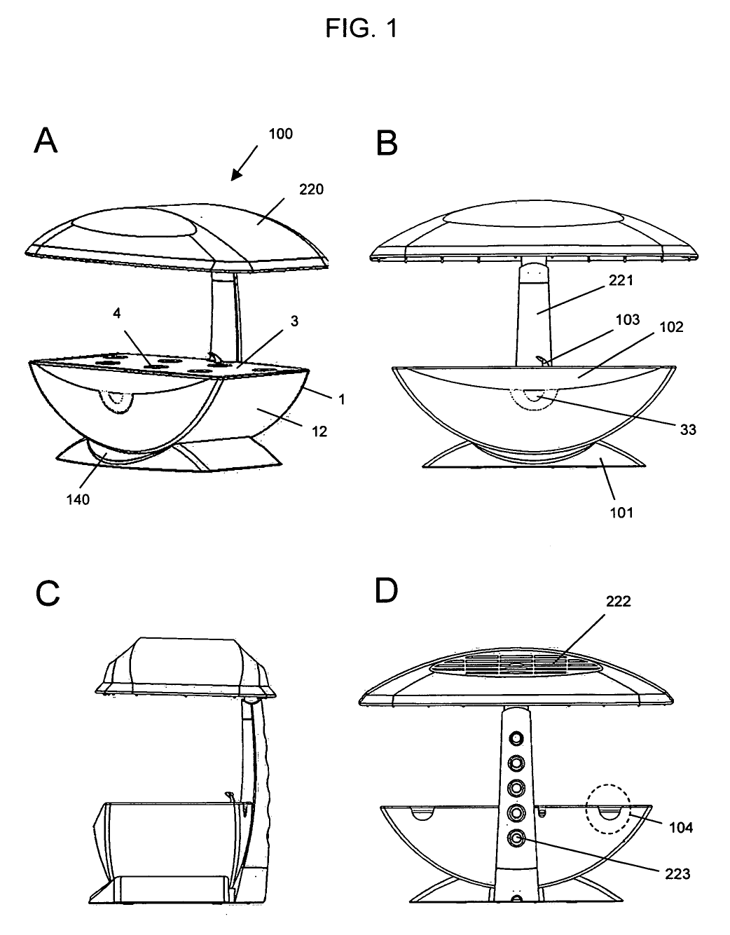

FIGS. 1A-D are illustrations showing a perspective view, a front view, a side view, and a back view, respectively, of a device 100, for growing a plant or germinating a seed into a plant with a photoradiation apparatus and smart garden, of this invention. The device shown in FIGS. 1A-D includes a photoradiation apparatus for delivering artificial photoradiation with a base 101 and a notched arm 221 for changing the height 223 of the photoradiation hood 220 as the plants grow (not shown). The photoradiation hood 220 has vent holes 222 for heat produced by the bulbs (not shown) to escape. The cover 3 of the vessel 12 has seven openings 4 for plants. The plant-growing device 1 has door 102 for adding liquid and viewing roots. Below the door 102 is a liquid level gauge. In the cover 3 there is a nutrient inlet cover 103. The cover 3 has two tabs and the vessel has two cut-outs and indentations, which together serve as a means for lifting the cover 104. The smart garden's display panel 140 is shown in outline.

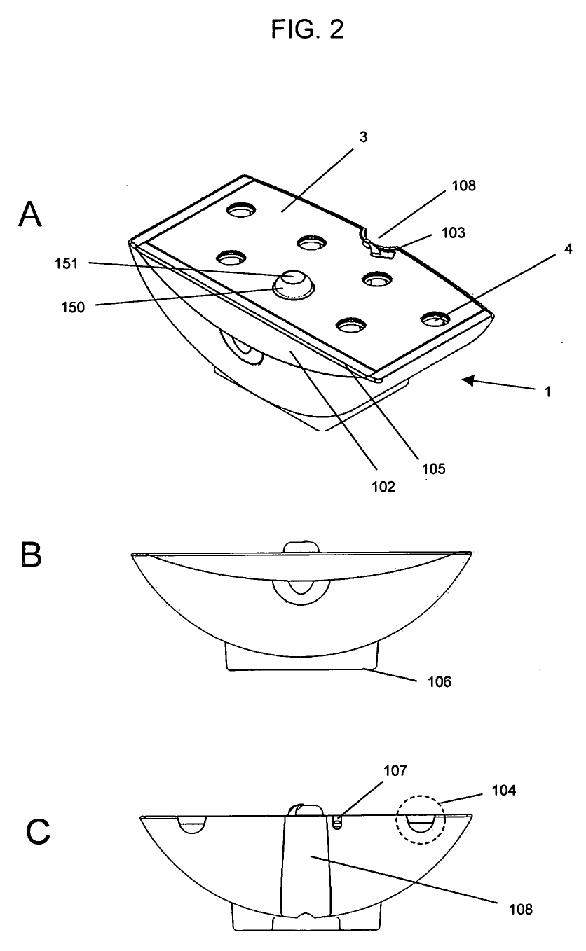

FIGS. 2A-C are illustrations showing a perspective view, a front view, and a back view, respectively, of a device 1 of this invention, for growing a plant or germinating a seed into a plant. One germination cap 150 having a lens 151 is shown over one of the openings 4 in the cover 3. There is a nutrient inlet cover 103 in the cover 3. The device is shaped to have an indentation 108 for a photoradiation apparatus arm (not shown). The door 102 is attached to the device with a hinge 105. The device 1 has a flat bottom surface 106 and requires no additional support means. FIG. 2C shows an optional power cord exit 107. If the device is to be set in a base (not shown), the electricity can be supplied to the device 1 directly into the base, with the power cord entering the base, with indirect, easily detachable, electric connections linking the base and the device 1 and providing electricity to the device. The connections could pull apart upon lifting the device 1 up off the base, such as when performing maintenance.

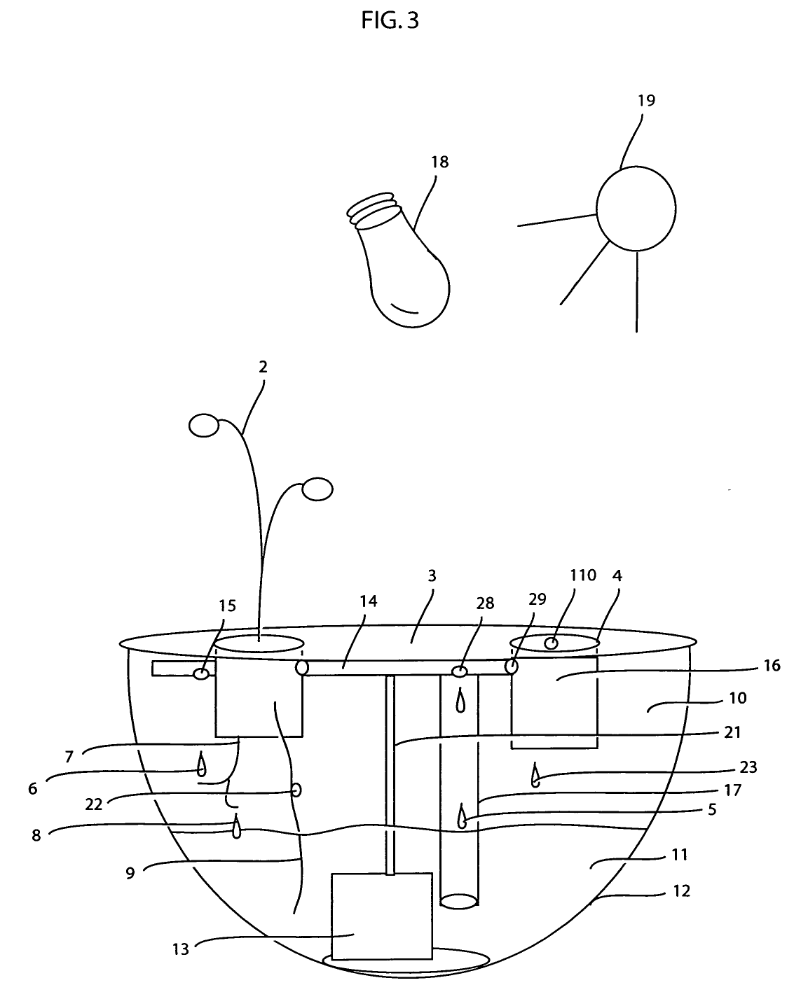

FIG. 3 is an illustration showing a front longitudinal cross-section perspective view of a device 1, for growing a plant or germinating a seed into a plant, of this invention. A plant 2 grows out of a plant support 16 that is frictionally engaged in an opening 4 in a cover 3 on a vessel 12. The vessel 12 contains a liquid 11 and a gas 10. A pump 13 rests on the bottom of the vessel 12 in the liquid 11 and is connected to a conduit 14 by a tube 21. The conduit 14 also contacts the plant supports 16. Roots 7 from the plant grow in the gas 10 and roots 9 in the liquid 11. The conduit 14 has exits 15 for liquid to drop and contact the plant support 16, contact the plant 2, and fall in drops 23 from the plant support 16 into the liquid 11 or descend in drops 22 along a root 9 into the liquid 11. Drops 5 fall directly into the liquid 11 from a drop height after exiting the conduit 14, or drops 6 fall onto roots 7 after exiting the conduit 14, and off of the roots 8 into the liquid 11 after delivery to the plant 2. A drop guard 17 ensures that some of the drops 5 fall directly into the liquid. Photoradiation is optionally provided from an artificial source 18 or a natural source 19. A wick (not shown) can optionally be placed in contact with the plant support 16 and the liquid 11 in case of a power outage.

In an embodiment of this invention, the vessel 12 shown in FIG. 3 is partially filled with liquid 11 comprising water and nutrients. A pump 13 is placed in the liquid 11. A cover 3 having a conduit 14 on the lower side that has openings 4 for suspending two plants is placed on the vessel 12, after the conduit 14 is connected to the pump 13 by a tube 21. A plant support 16 is placed in the opening 4 in the cover. Gas 10 comprising oxygen gas, typically air, is above the liquid 11. Seeds (not shown) are placed on or in the plant support 16. The device 1 is placed in a gas comprising carbon dioxide gas and oxygen gas, typically air. Photoradiation is provided from an artificial 18 or natural source 19. Liquid 11 is delivered from the pump 13 through the conduit 14. A first portion of the liquid falls in drops 5 out of exits 15 in the conduit 14 and into the liquid 11. A second portion of liquid is delivered 20 through exits 15 to the plant support 16 and the seed and falls in drops 23 from the plant support 16 into the liquid 11. After the passage of time, the seeds germinate. Roots 7 of the plants 2 grow through and out of the plant support 16 into the gas 10. The second portion of liquid also descends in drops 22 along roots 7 growing in the gas and falls in drops 8 into the liquid 11 or descends along directly into the liquid 11 on roots 9 that have grown into the liquid 11. A third portion of liquid falls in drops 6 onto roots. An optional drop guard 17 ensures that some drops 5 always drop directly into the liquid 11 after the roots have grown throughout much of the vessel 12. Optionally the drop guard 17 has humidity holes (not shown) which are permeable to humidity but not roots. Oxygen is delivered to the plants in at least five ways: 1) delivery 20 at about or below the plant transition region from the liquid 11 exiting the conduit 14, 2) to the roots 7 growing in the gas 10, 3) to the roots 9 growing in the gas 10 from the drops 6 falling on the roots, 4) to the roots 9 growing in the liquid 11 from the dissolved oxygen in the liquid by diffusion from the gas 10 and from the drops 5 and 8 falling into the liquid 11 which increase the dissolved oxygen, and 5) to the roots 7 growing in the gas 10 from the humidity increased by the drops 5 and 8 falling into the liquid 11. The liquid level is maintained at a level high enough for the pump 13 to deliver liquid through the conduit 14 to the plants 2, and at a level low enough to allow the roots 7 and 9 to obtain oxygen from the gas 10, particularly if air roots have developed. Preferably the liquid level is low enough for the first portion of liquid to fall in drops. A greater drop height may result in more oxygen being dissolved in the remaining liquid. The liquid level is checked weekly at first, then more often as the plants become larger and utilize more liquid. Water and/or nutrients are added as necessary.

The seeds germinate and grow into plants. The liquid level is monitored using an optional liquid level gauge (not shown). As necessary, liquid is added using a liquid inlet (not shown). Nutrients are added in the nutrient inlet (not shown) every other week. As the plants grow, selected tissues are harvested. Each month, the liquid is optionally emptied using the pump through a liquid exit tube (not shown). When harvest is complete, the plants are removed and the device 1 is disassembled and cleaned.

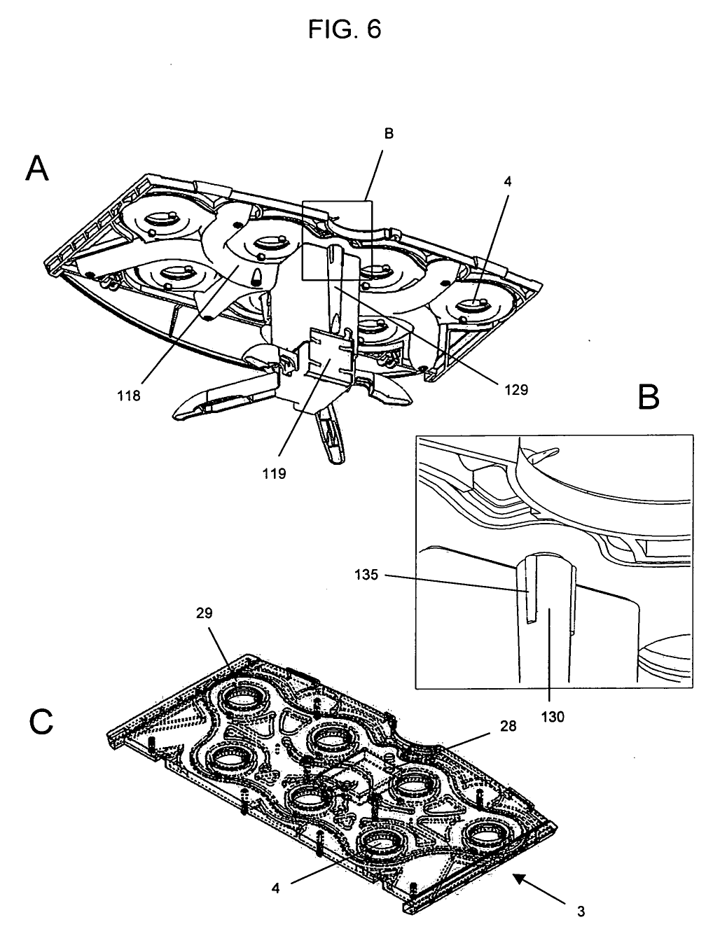

When a device of this invention, parts of which are shown in FIGS. 1-13, is in use, the liquid flows in the through the conduit 14 shown in FIG. 3. Liquid is pumped from inside the vessel 12 up the tube 21 that connects to the cover 3 in the center and flows through the conduits 14 to the exits 15. Optionally the cover comprises a lower and an upper cover that together form the conduit(s) 14. The liquid can exit the conduits 14 at an acceleration equal to or greater than 9.8 m/s². In an embodiment, the liquid is delivered to each plant separately. The liquid optionally exits as a stream or as visually distinguishable drops. As shown in FIG. 6C, the first portion of liquid exits 28 and falls on the remaining liquid inside the vessel 12 or a root of a plant growing in the device. The second portion of liquid exits 29 a conduit 14 and contacts a plant growth medium or support (not shown) and/or a plant, then falls or descends down a root to a terraced aerator or into the remaining liquid.

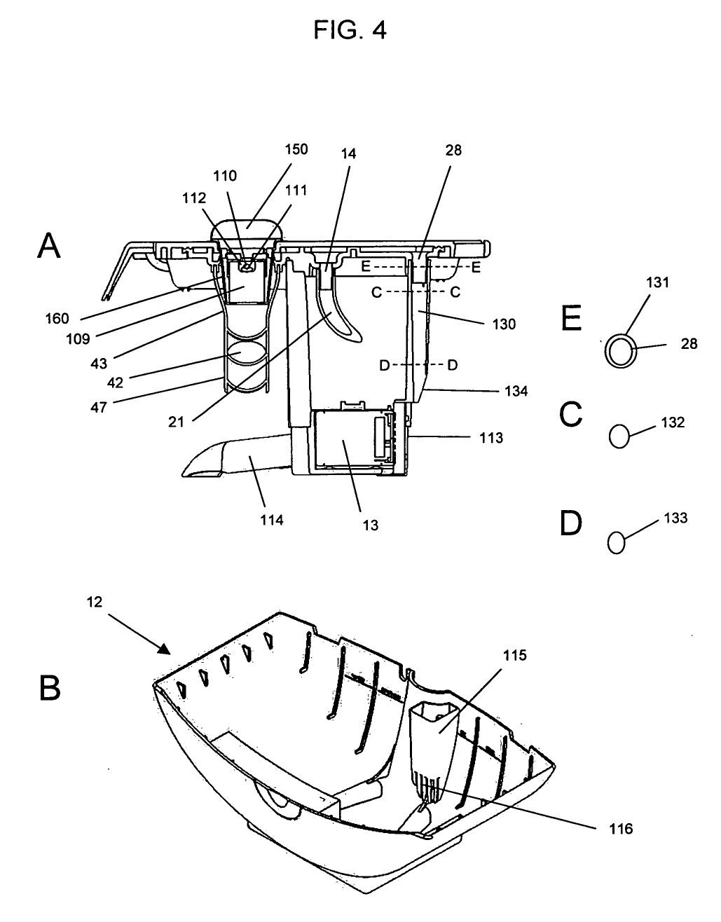

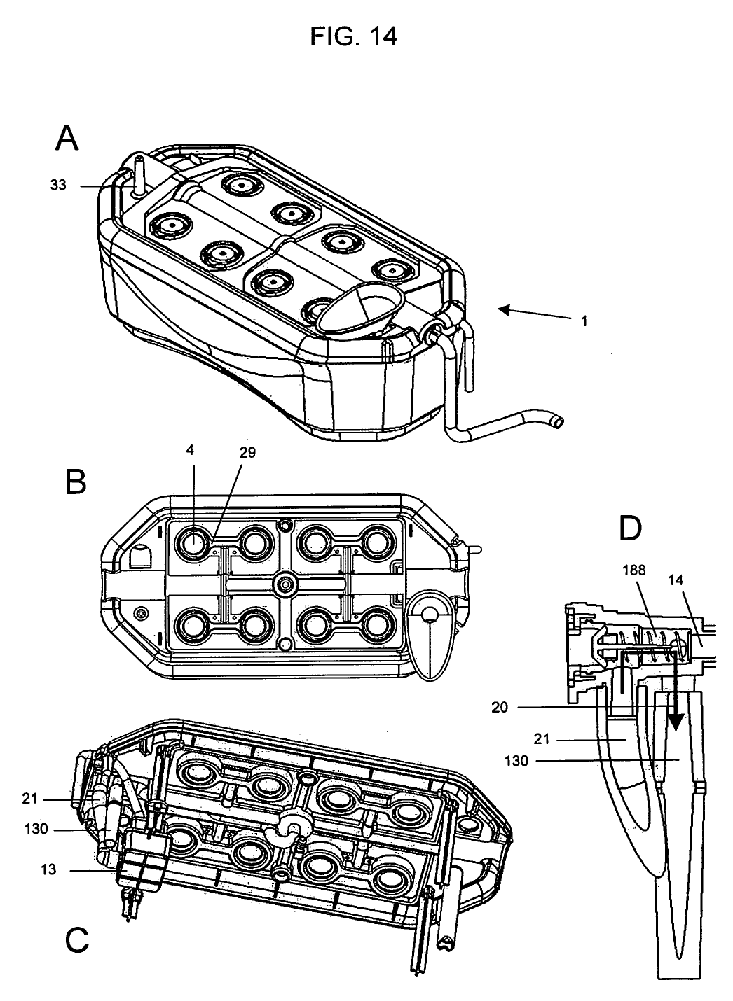

FIG. 4A is an illustration showing a side longitudinal cross-section of the upper portion, including the cover (not labeled), a germination cap 150, a seed-support medium (not labeled), downdraft venturi 130, pump 13, and a portion of the cover stand, of the device shown in FIGS. 2A-D. One of several legs 114 of the stand (not labeled) is shown which support the liquid delivery components and the cover in side the vessel. The pump 13 delivers liquid up a tube 21 to a conduit 14 in the cover, which can be seen in FIG. 14C. The tube 21 is not shown connecting to pump 13 in FIG. 4A because the connection is outside of this section. A first portion of the liquid is directed along the conduit and delivered to a first portion exit 28 which delivers the liquid to a downdraft venturi 130 and out a venturi exit 134 to a reservoir liquid that would be in the vessel. The gas above the reservoir liquid contains oxygen, therefore, as the liquid falls through the downdraft venturi, the concentration of dissolved oxygen in the first portion of liquid is increased or maintained. During use, the level of the reservoir liquid fluctuates between at about the venturi exit 134 to below the seed support medium. After the liquid falls through the venturi to the reservoir liquid, reservoir liquid enters the pump 13 through the pump inlet 113. A second portion of liquid in the conduit 14 is delivered to a one or more second portion exits (not shown) for delivery to the seeds 110 or plants. The second portion of liquid enters a net basket 160 at a net basket inlet (not shown), flows along one or more channels (not shown) in the net basket 160, contacts a growth medium 109, and contacts a seed 110 resting in a dibble 112 and attached with an adhesive 111. The liquid in the growth medium is substantially prevented from evaporating through the opening (not shown) by a germination cap 150. After contacting the seed 110 or growth medium 109, the liquid falls through the gas between the seed support medium and the reservoir liquid level inside the device, and may fall directly to the reservoir liquid, contact a plant root, or contact a terraced aerator 47. This terraced aerator 47 in FIG. 4A has three terraces 42 attached to the cover by a terrace support means. The configuration of the terraces 42 is selected to enable about all drops or streams falling from the seed support medium to contact the uppermost terrace. After the liquid contacts the uppermost terrace, the liquid falls in drops or streams optionally to the next terrace(s) or to the reservoir liquid depending on the level of the reservoir liquid.

FIGS. 4C-E are illustrations showing horizontal cross-section views of the downdraft venturi tube at various heights. FIG. 4E shows the exit for the first portion 28 emptying into the upper tube portion of the downdraft venturi 130 having a first cross-sectional area 131. FIG. 4C shows a second cross-sectional area 132 that is smaller than the first 131, and FIG. 4D shows a third cross-sectional area 133 that is even smaller.

FIG. 4B is an illustration showing a perspective view of the lower potion, including the vessel 12 and nutrient basket 115, of the device shown in FIGS. 2A-D. The nutrient basket 115 has nutrient basket outlets 116.

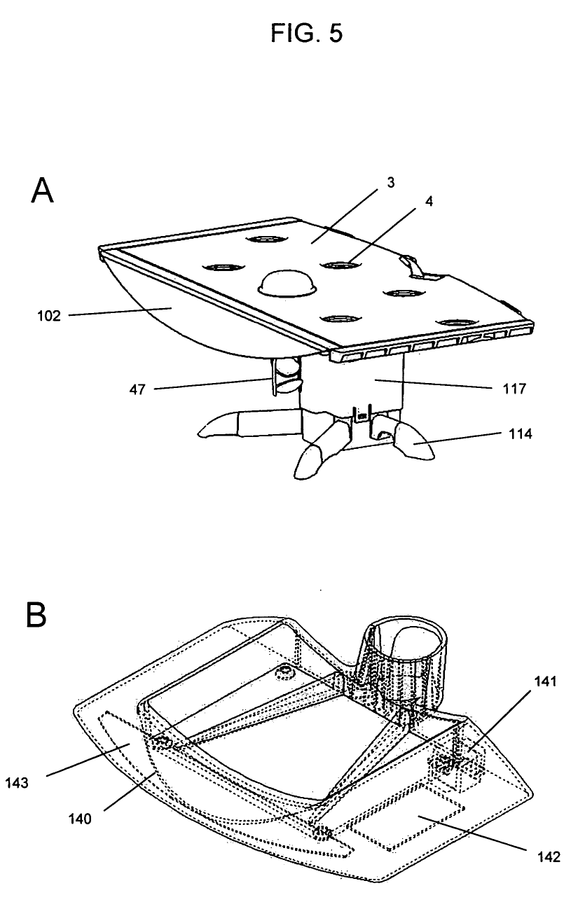

FIG. 5A is an illustration of a top perspective view of the portion of the device shown in FIG. 4A. FIG. 5A shows a six uncovered plant openings 4 and one covered by a germination cap (not labeled). The door 102 is attached to the cover 3, and the stand 117, of which three legs 114 are visible. One terraced aerator 47 is visible. FIG. 5B is an illustration of a perspective ghost view (dashed lines) of the base, including the smart garden, of the device shown in FIGS. 1A-D. The base is an optional support for a device of this invention and a photoradiation apparatus (not shown). This base contains a smart garden display panel 140 which also serves as a data entry panel. Behind the panel 140 is a circuit board controller 143 for the smart garden device. The side of the base contains the circuit board for the electric power 142 of the device which is connected to a transformer 141.

FIG. 6A is an illustration of a lower perspective view of the portion of the device shown in FIGS. 4A and 5A. This view of the portion of the device shows plant openings 4, the filter 119 for the liquid entering the pump at the pump inlet, optional internal cover support arms 118, and the aspirator 129, which is also a downdraft venturi. FIG. 6B is a detail illustration of the box in FIG. 6A, showing the downdraft venturi 130 and the gas inlets 135.

FIG. 6C is a bottom perspective ghost view of the cover 3 shown in FIG. 6A. Plant openings 4, the exit for the first portion of liquid 28 and exits for the second portion of liquid 29 are labeled. The inside of the cover 3 is configured with islands for directing the liquid flow to each opening 4 through the second portion exits 29 and through the first portion exits in a selected ratio.

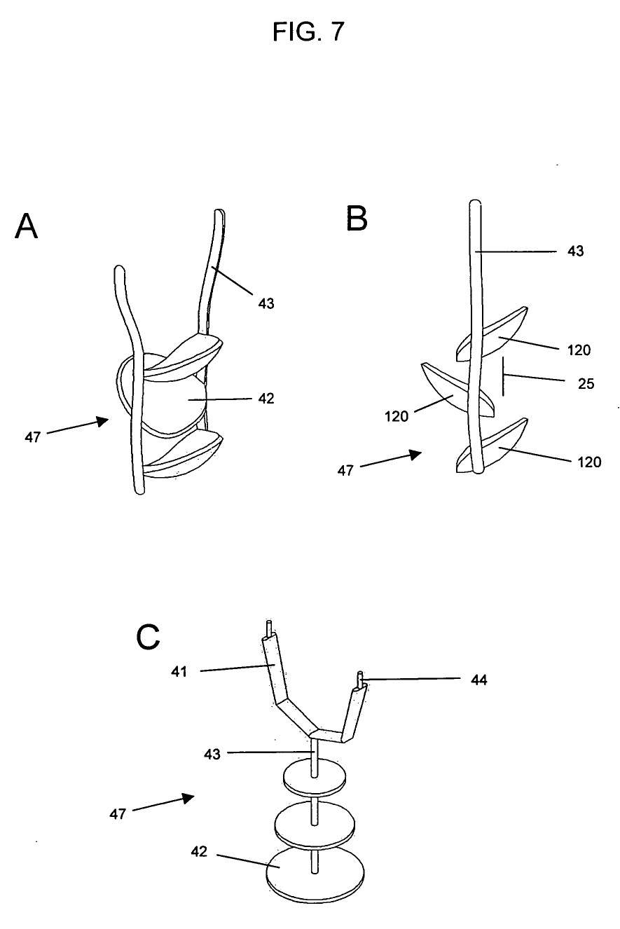

FIGS. 7A-B are illustrations of a perspective view of a terraced aerator 47 with curved, liquid-retaining, alternating terraces; a side view of a terraced aerator having flowform terraces, and a perspective view of an terraced aerator having coaxial flat terraces. FIG. 7B shows a terraced aerator 47 having flowform terraces 120. Liquid falls a drop height 25 from a first terrace to a second terrace. The flowforms 120 are supported by a terrace support means 43. The flowforms direct the liquid to emulate the swirls and vortices of a mountain stream. These currents of the liquid enable oxygen in the gas surrounding the flowform 120 to mix with the liquid thereby further increasing or maintaining the concentration of dissolved oxygen in the liquid, in addition to the increase or maintenance of dissolved oxygen concentration resulting from the drop or stream falling a drop height 25. Three flat terraces 42 in the terraced aerator shown in FIG. 7C are in fixed positions on a terrace support means 43 which projects through the centers of the round terraces 42. The terrace support means is connected to the liquid directing means 41 which is connected to the terrace aerator suspending means 44, which is two clips that are removably connectable to a cover (not shown) of a device (not shown) of this invention. When liquid contacts the first (uppermost) terrace 42, a portion can fall in drops a drop height distance before contacting the second terrace (middle) 42. A portion of the liquid can optionally adhere by capillary action to the lower side (not shown) of the first terrace 42, descend along the terrace support means, and contact the second terrace 42 without falling in drops.

In a terraced aerator in which the terraces have at least portions of side walls, the terrace walls and openings can cause a terrace to contain a portion of liquid causing drops falling from the higher terrace to fall into the contained liquid and increase the dissolved oxygen concentration of the liquid. The drop height distance is optionally selected to produce a desired sound decibel level upon contact of a drop from an upper terrace with a contained portion of liquid and/or a liquid reservoir (not shown) below the lowest terrace. Optionally terraces are arranged by increasing diameter from the top down to ensure that all liquid contacting the first terrace contacts the second next lower terrace and that all liquid contacting the second terrace contacts the third terrace, etc.

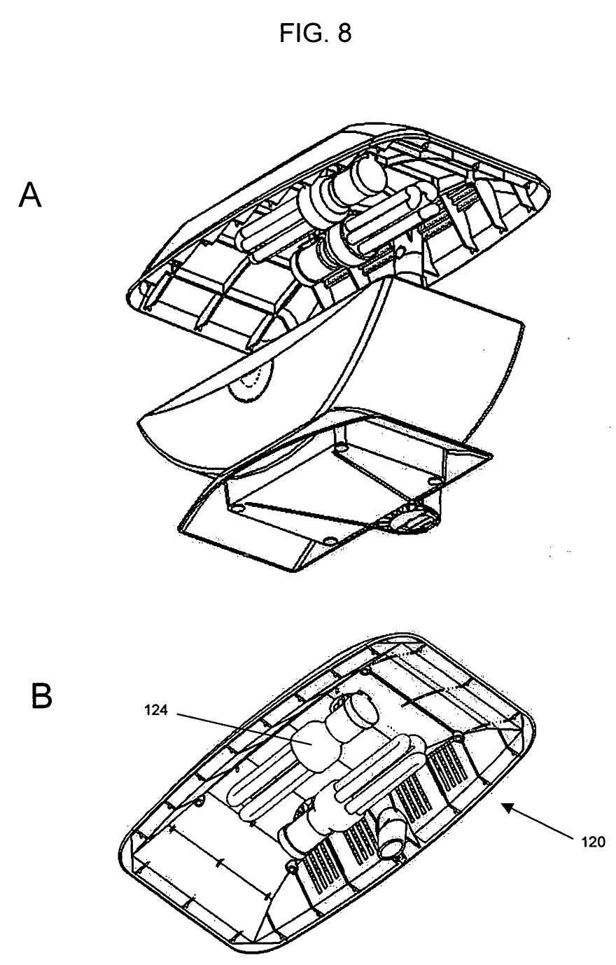

FIG. 8A is an illustration of a bottom perspective view of the device shown in FIGS. 1A-D. FIG. 8B is a bottom perspective view of the artificial photoradiation hood 120 of the device shown in FIG. 8A, showing two artificial photoradiation sources 124.

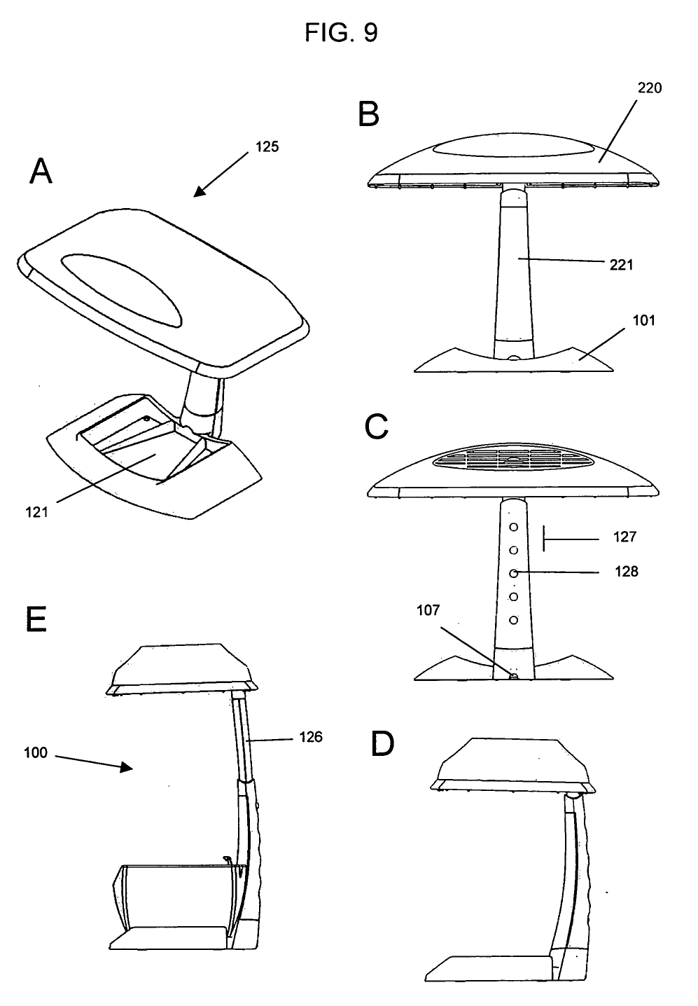

FIGS. 9A-E are illustrations showing a perspective view, a front view, a back view, a side view, and a side view with the arm extended, respectively, of the photoradiation apparatus 125 shown in FIGS. 1A-D. FIG. 9A shows a device or vessel receiving means 121. FIG. 9B shows the base 101, adjustable photoradiation arm 221, and photoradiation hood 220. FIG. 9C shows the arm extension notches 128 and the height of an extension 127. A power cord exit 107 is also visible. FIG. 9E shows the device 100 with the arm completely extended 126.

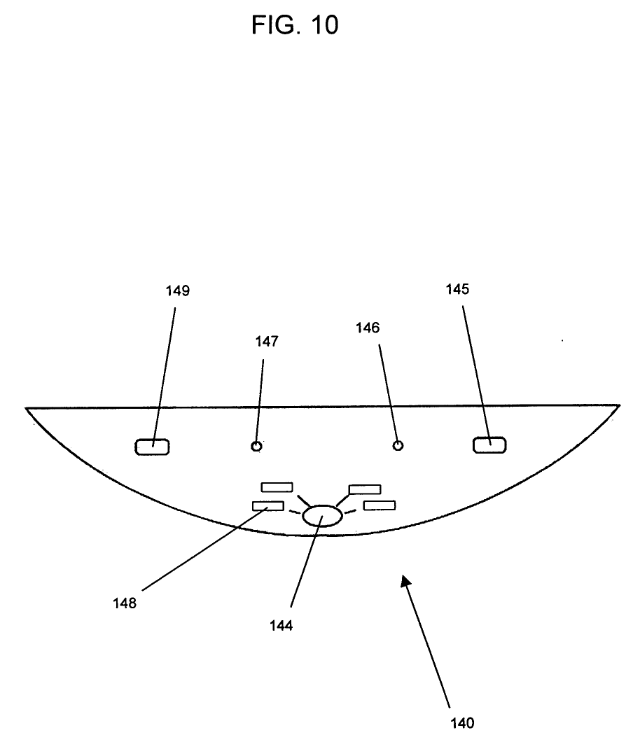

FIG. 10 is an illustration showing a front view of a smart garden display panel 140 of this invention. The panel 140 contains a means for inputting photoradiation cycle override data 149, a means for alerting a user to add liquid 147 to the device, a means to alert a user to add nutrient 146 to the device, and means for inputting nutrient cycle reset data 145. Optionally the adding liquid 147 and adding nutrient 146 signal means flash a light to alert a user. The panel 140 also has a timing cycle selection input and display means 144. This data is used to select the cycle of the pump and/or the photoradiation apparatus. The cycle selected can be displayed by a lighting up the name 148 of the selected cycle.

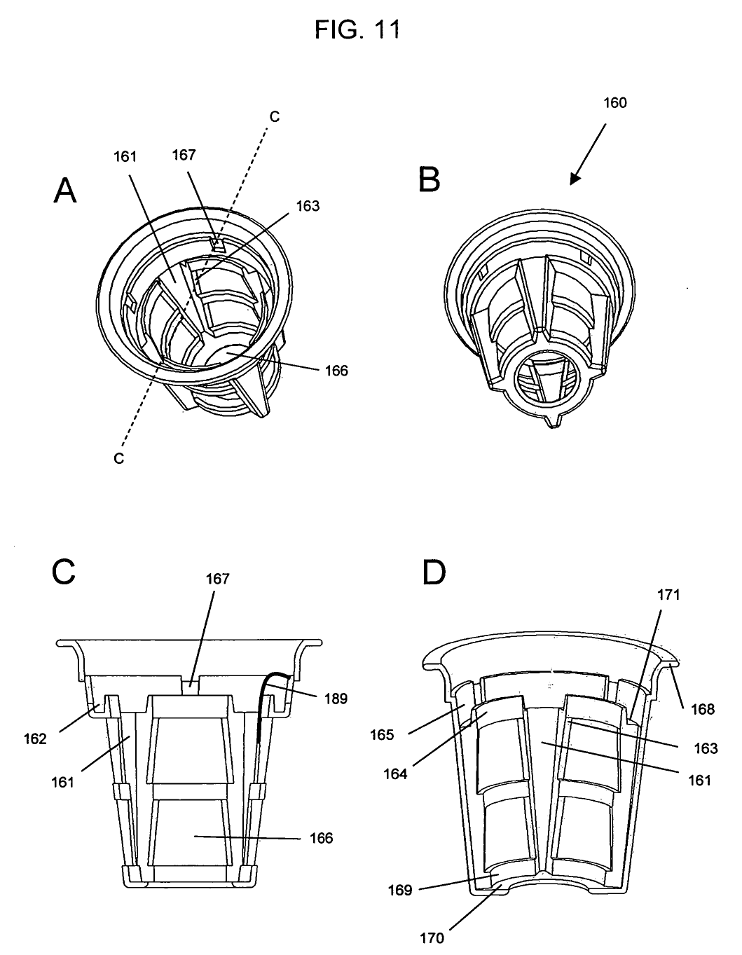

FIGS. 11A-D are illustrations showing a top perspective view, a bottom perspective view, a longitudinal cross-sectional view, and a cut-away top perspective view, respectively, of a net basket 160 of this invention. The section for FIG. 11C is marked in FIG. 11A, and the cut-away of FIG. 11D is at the same section as FIG. 11C. FIG. 11A shows a net basket liquid inlet 167, a channel with a vertical component 161, a proximal edge 163 of the channel 161, and a net basket hole 166 in the bottom for roots of a plant to grow out and/or liquid to exit. FIG. 11C shows a net basket liquid inlet 167, a channel having a horizontal component 162, a channel having a vertical component 161, and a net basket hole 166 in the side. FIG. 11D shows the U-shaped channel having a horizontal component 171, the proximal wall 164 of the channel 171, a basket support means 168, the proximal side of the distal wall 165, a channel having a vertical component 161, the proximal edge of a wall of the channel 161, and the side 169 and bottom 170 of an L-shaped horizontal channel. A cross-section line of an optional proximal wall 189 that could seal the upper portion of the horizontal channel is shown.

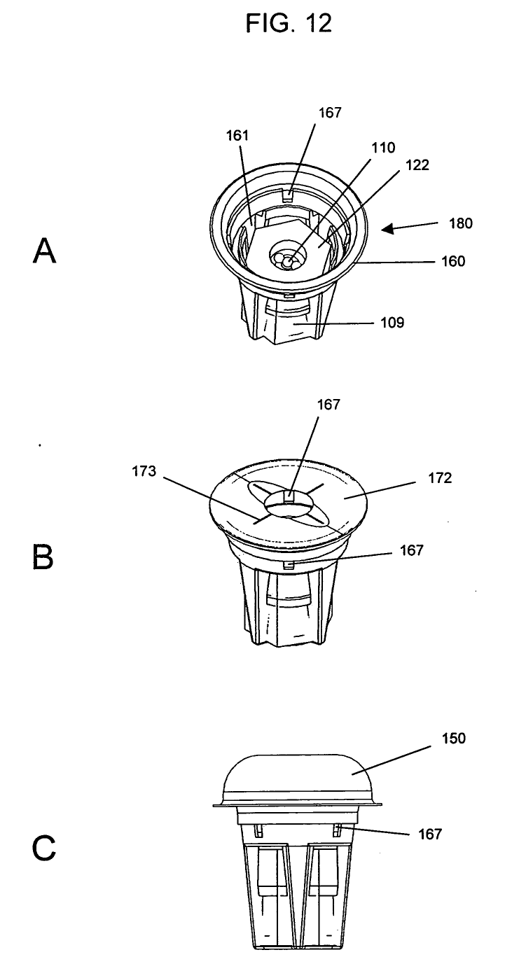

FIGS. 12A-B are illustrations showing a top perspective views of a seed-support medium 180 of this invention. FIG. 12A shows the net basket 160 shown in FIGS. 11A-D. The net basket liquid inlet 167, channel having a vertical component 161, upper surface 122 of the growth medium 109, and the seeds 110 are visible. FIG. 12B shows a label 172 with slits 173, and liquid inlets 167. FIG. 12C shows the seed-support medium shown in FIG. 12B with a germination cap 150. The liquid inlets 167 are visible.

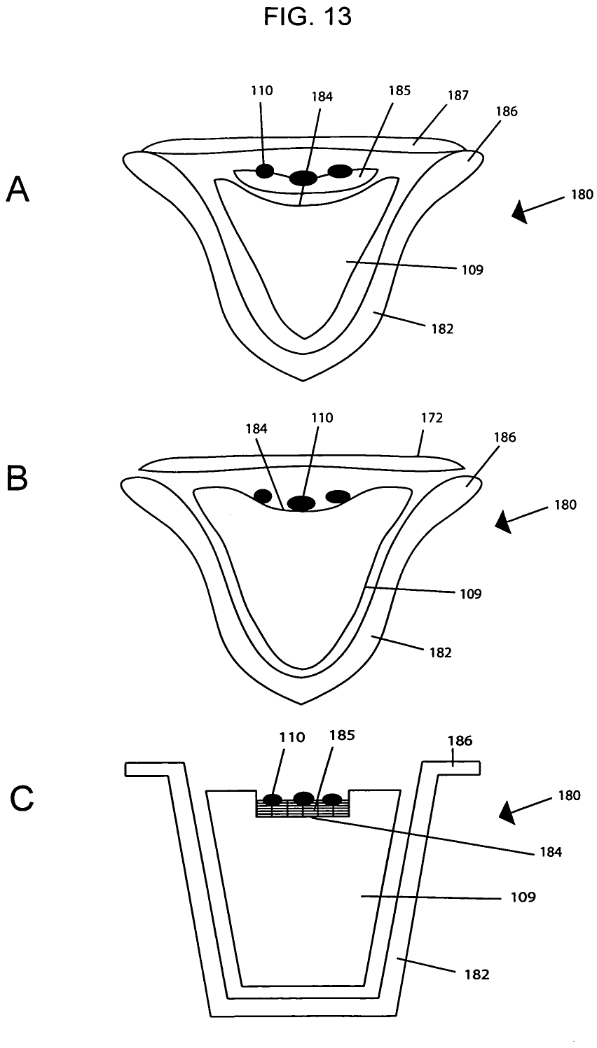

FIGS. 13A-C are illustrations showing longitudinal cross-sectional views of seed-support media of this invention. FIG. 13A shows a rigid, modular receptacle 182 with a support means 186, containing a growth medium 109 which has a concave recess 184. The seed support medium is covered by a seal 187. The seeds 110 are not in a seed-bearing substrate. FIG. 13B shows a rigid, modular receptacle 182 with a support means 186, containing a growth medium 109 which has a concave recess 184. The seed support medium is covered by a label 172. FIG. 13C shows a rigid, modular receptacle 182 with a support means 186, containing a growth medium 109. The seeds 110 are in a seed-bearing substrate 185.

FIG. 14A is an illustration showing a top perspective view of an alternative hydroponics device 1 of this invention, showing a liquid level gauge 33. FIG. 14B is an illustration showing a top view of the lower cover of the device show in FIG. 14A. An exit for the second portion 29 to the plant opening 4 is shown. FIG. 14C is an illustration showing a bottom perspective view of the lower cover of the device show in FIG. 14A. The pump 13, tube 21 from which the liquid leaves the pump 13 to the cover, and the downdraft venturi 130 are labeled. FIG. 14D is an illustration showing a detail cross-sectional view of the tube 21, valve 188, and venturi 130 are shown. The arrow 20 shows the delivery pathway of the first portion of liquid. A valve 188 directs water to the conduit 14 for the second portion of liquid and to the downdraft venturi 130 for the first portion of liquid. In FIG. 14D, the downdraft venturi appears to not be open on the bottom because the venturi is at an angle relative to the cross-sectional plane, but the venturi is configured to allow the liquid to fall directly to the reservoir liquid that would be in the vessel (not shown).

This invention provides a device for growing a plant or germinating a seed into a plant, wherein the plant may have one or more roots, the device comprising: a vessel for containing a liquid; a means for removably suspending the plant in a gas above the liquid; a means for elevating a first portion of the liquid above the remaining liquid in the vessel and into the gas wherein the first portion of liquid falls through the gas into the remaining liquid; and a means for contacting a second portion of the liquid with the plant, seed, or a growth medium contacting the plant or seed and allowing the second portion of liquid to return to the remaining liquid; whereby the one or more roots are permitted to grow in the gas and in the remaining liquid.

In an embodiment, the means for contacting the second portion of liquid with the plant, seed, or growth medium comprises delivering the second portion of liquid through a channeled net basket. In an embodiment, the means for elevating and/or means for delivering comprise a conduit.

This invention provides a device for growing a plant or germinating a seed into a plant, wherein the plant has one or more roots, the device comprising: a vessel for containing a liquid; a means for removably suspending the plant in a gas above the liquid; a conduit in fluid communication with the liquid and the gas; and a means for delivering a first portion and a second portion of the liquid through the conduit whereby the first portion of liquid falls through the gas into the remaining liquid in the vessel and the second portion of liquid contacts the plant, seed, or a growth medium contacting the plant or seed, and descends into the remaining liquid; whereby the one or more roots are permitted to grow in the gas and in the remaining liquid.

In an embodiment, the first portion of liquid falls in drops or streams. In an embodiment, the drops have diameters greater than about 200 microns, greater than about 350 microns, greater than about 500 microns, greater than about 1000 microns, greater than about 2000 microns, or greater than about 5000 microns. In an embodiment, the conduit is also in fluid communication with the liquid and the plant, seed, or growth medium contacting the plant.

In an embodiment, the device further comprises a means for delivering a third portion of the liquid through the conduit whereby the third portion of liquid falls through the gas, is permitted to contact the one or more roots, and contacts the remaining liquid. In an embodiment, the device is for growing more than one plant.

In an embodiment, the second portion of liquid contacts the plant, seed, or the growth medium at about or below the height of the seed or transition region of the plant. In an embodiment, the device comprises a means for delivering the second portion of liquid to each of a plurality of plants separately. In an embodiment, the first portion of liquid only contacts the gas and the remaining liquid. In an embodiment, the conduit has separate first and second exits for the first and the second portions of liquid. In an embodiment, the means for delivering a first portion and a second portion of said liquid comprises a pump. In an embodiment, the first portion of liquid is delivered substantially vertically downward. In an embodiment, the first portion of liquid falling through the gas into the remaining liquid increases the dissolved oxygen content of the remaining portion of liquid and/or the first portion of liquid. In an embodiment, the first portion of liquid falling into the remaining liquid increases negative ions within the device. In an embodiment, the liquid and the one or more roots are completely contained in one vessel.

In an embodiment, the device further comprises a means for intermittently delivering the first and second portions of liquid. In an embodiment, the intermittently delivering comprises an on cycle and an off cycle wherein the on cycle is about twice as long as the off cycle.

In an embodiment, the device further comprises a means for delivering photoradiation to the plant, seed, or cutting. In an embodiment, the device further comprises a downdraft venturi.

In an embodiment, the device further comprises a means for dampening the sound produced when the first or the second, or both portions of liquid descend into the remaining liquid. In an embodiment, the means for dampening sound produced by the second portion of liquid descending comprises a terraced aerator. In an embodiment, the means for dampening sound comprises a terraced aerator comprising one or more terraces; and a means for suspending the terraced aerator below a portion of the plant or a growth medium contacting the plant in the gas above the liquid; wherein the second portion of liquid contacts the plant or the growth medium and descends to the first terrace, then descends from the first terrace into the remaining liquid. In an embodiment, the means for dampening sound produced by the first portion of liquid descending comprises an enclosure for the descending first portion of liquid. In an embodiment, the device also comprises a terraced aerator comprising: two or more terraces; and a means for suspending the first terrace above the second terrace; and a means for suspending the terraced aerator below a portion of the plant or a growth medium contacting the plant in the gas above the liquid; wherein the second portion of liquid contacts the plant or the growth medium and descends to the first terrace, then descends from the first terrace to the second terrace, and then descends from the second terrace into the remaining liquid. In an embodiment, the second portion of liquid descends from the first terrace to the second terrace or from the second terrace to the remaining liquid, or both, in drops or streams. In an embodiment, the liquid descending in drops or streams to the second terrace or descending into the remaining liquid produces a sound of less than about 57 decibels.

This invention provides a cover comprising: a means for removably suspending a plant in a gas above a liquid in a vessel; a means for elevating a first portion of the liquid above the remaining liquid in the vessel and into the gas wherein the first portion of liquid falls through the gas into the remaining liquid; and a means for contacting a second portion of the liquid with the plant, seed, or a growth medium contacting the plant or seed and allowing the second portion of liquid to return to the remaining liquid; whereby the one or more roots are permitted to grow in the gas and in the remaining liquid. Elements of the cover can be housed in a stand for supporting the cover.

This invention provides a method for growing a plant or germinating a seed into a plant comprising: providing a device of this invention; delivering a first portion and a second portion of the liquid through the conduit whereby the first portion of liquid falls through the gas into the remaining liquid in the vessel and the second portion of liquid contacts the plant, seed, or a growth medium contacting the plant or seed, and descends into the remaining liquid; and providing nutrients, carbon dioxide, oxygen, and light to the plant; whereby the plant grows and a root of the plant is permitted to grow in the gas and in the remaining liquid.

This invention provides a kit for growing a plant comprising a device of this invention and instructions for using the device. This invention provides a kit for growing a plant or germinating a seed into a plant, the kit comprising: a device for growing a plant or germinating a seed into a plant wherein the plant has one or more roots comprising: a vessel for containing a liquid; a means for removably suspending the plant in a gas above the liquid; a conduit in fluid communication with the liquid and the gas; and a means for delivering a first portion and a second portion of the liquid through the conduit whereby the first portion of liquid falls through the gas into the remaining liquid in the vessel and the second portion of liquid contacts the plant, the seed, or a growth medium contacting the plant or seed, and descends into the remaining liquid; whereby the one or more roots are permitted to grow in the gas and in the remaining liquid; and instructions for using the device.

In an embodiment, the kit also comprises one or more components selected from the group consisting of: terraced aerators, downdraft venturis, net baskets, germination caps, sets of germination caps, seed support media, and smart garden devices.

This invention provides a method for growing a plant or germinating a seed into a plant, wherein the plant has at least one root, the method comprising: providing a vessel for containing a liquid; providing a means for removably suspending the plant in a gas above the liquid; providing a conduit in fluid communication with the liquid and the gas; and providing a means for delivering and delivering a first portion and a second portion of the liquid through the conduit whereby the first portion of liquid falls through the gas into the remaining liquid in the vessel, and whereby the second portion of liquid contacts the plant, the seed, or a growth medium contacting the plant or seed, and descends into the remaining liquid; whereby the root of the plant is permitted to grow in the gas and in the remaining liquid.

In an embodiment, the means for delivering and delivering the second portion of liquid through the conduit whereby the second portion contacts the plant, the seed, or the growth medium comprises: providing a net basket for supporting the plant or seed, the net basket comprising a liquid inlet and a channel having a vertical component for transporting the liquid; delivering the liquid from the conduit to the liquid inlet; transporting the liquid through the liquid inlet to the channel having a vertical component; transporting the liquid through the channel having a vertical component; and contacting the plant or seed with the liquid; wherein the plant grows and wherein one or more roots of the plant and the liquid are allowed to exit through one or more holes in the net basket.

In an embodiment, the first portion of liquid falls in drops or streams. In an embodiment, the drops have diameters greater than about 200 microns, greater than about 350 microns, greater than about 500 microns, greater than about 1000 microns, greater than about 2000 microns, or greater than about 5000 microns. In an embodiment, the conduit is also in fluid communication with the liquid and the plant or a basket or growth medium contacting the plant.

In an embodiment, the method further comprises delivering a third portion of the liquid through the conduit whereby the third portion of liquid falls through the gas, contacts the one or more roots, and contacts the remaining liquid. In an embodiment, the second portion of liquid contacts the plant or growth medium at about or below the height of the transition region of the plant or at about the seed.

In an embodiment, the method is for more than one plant or seed. In an embodiment, the delivering is performed by pumping. In an embodiment, the pumping is performed while the plant or the seed is germinating. In an embodiment, the delivering comprises the first portion of liquid exiting the conduit substantially vertically downward.

In an embodiment, the method comprises delivering the second portion of liquid to each plant, seed, or cutting separately. In an embodiment, the first portion of liquid only contacts the gas and the remaining liquid. In an embodiment, the conduit has first and second exits and the method further comprises delivering the first and second portions of liquid through the first and second exits.

In an embodiment, the method further comprises increasing the dissolved oxygen content of the first and remaining portions of liquid when the first portion of liquid falls through the gas into the remaining liquid. In an embodiment, the method further comprises increasing the negative ions within the vessel when the first portion of liquid falls into the remaining liquid. In an embodiment, the method comprises continuously delivering the first and second liquid portions.

In an embodiment, the method further comprises adding additional liquid to the device wherein the additional liquid is above pH 5.5. In an embodiment, the method comprises containing the liquid and all of the one or more roots in one vessel.

In an embodiment, the second portion of liquid contacts the plant or the seed and then descends to a first terrace of a terraced aerator, then descends from the first terrace to a second terrace of a terraced aerator, and then descends from the second terrace into the remaining liquid.

This invention provides a method for delivering oxygen to a plant or seed which will germinate into a plant, the method comprising: providing a plant with at least one root or a seed which will germinate into a plant having at least one root; providing a liquid capable of having oxygen dissolved therein; providing a gas comprising oxygen gas; providing a means for elevating and elevating a portion of the liquid above the remaining liquid; allowing the portion of liquid to fall through the gas into the remaining liquid whereby oxygen gas dissolves in the portion of liquid or the remaining liquid thereby forming oxygenated liquid; and providing a means for contacting and contacting the plant or seed with the oxygenated liquid.

In an embodiment, the method further comprises providing a downdraft venturi and providing a means for allowing and allowing a second portion of liquid to descend through the downdraft venturi into the remaining liquid thereby increasing the dissolved oxygen content of the second portion of liquid or the remaining liquid. In an embodiment, the method step of allowing said portion of liquid to fall results in oxygen gas dissolving in the portion of liquid and the remaining liquid. In an embodiment, the dissolved oxygen content is increased in the second portion and in the remaining liquid. In an embodiment, the liquid falling through the gas into the remaining portion of liquid increases the humidity level of the gas.

In an embodiment, the method further comprises contacting the root with the humidity. In an embodiment, the method further comprises contacting the root with the gas comprising oxygen. In an embodiment, the method further comprises allowing the root to grow in the oxygenated liquid. In an embodiment, the method further comprises splashing the root with the oxygenated liquid. In an embodiment, after the portion of the oxygenated liquid falls through the gas and before the portion of liquid falls into the remaining oxygenated liquid, the portion of liquid contacts a terraced aerator.

This invention provides a method for increasing the dissolved oxygen concentration in a liquid within a hydroponics device comprising: providing a hydroponics device comprising: a vessel for containing a liquid; a means for removably suspending one or more of a plant, seed, a growth medium for contacting the plant or seed, and a net basket in a gas above the liquid; and a means for elevating a first portion and a second portion of the liquid above the remaining liquid and into the gas whereby the first portion of liquid falls through the gas into the remaining liquid in the vessel, and whereby the second portion of liquid can contact the plant, the seed, or a growth medium contacting the plant or seed, and descends into the remaining liquid; whereby the root of the plant is permitted to grow in the gas and in the remaining liquid; elevating the first portion of liquid above the remaining liquid and into the gas; elevating the second portion of liquid above the remaining liquid and into the gas; allowing the first portion of liquid to fall through the gas and into the remaining liquid; and allowing the second portion of liquid to contact the plant, seed, growth medium, or net basket and descend into the remaining liquid; whereby the dissolved oxygen concentration in the first portion of liquid, in the remaining liquid, or in both is increased.

In an embodiment, the hydroponics device further comprises a terraced aerator, wherein after contacting the plant, seed, growth medium, or net basket, the second portion of liquid contacts the terraced aerator before descending into the remaining liquid. In an embodiment, the hydroponics device is enclosed. In an embodiment, the method further comprises providing a downdraft venturi and providing a means for allowing and allowing a third portion of liquid to descend through the downdraft venturi into the remaining liquid thereby increasing the dissolved oxygen content of the third portion of liquid or the remaining liquid, or both.

This invention provides a hydroponics device for growing a plant or germinating a seed, the device comprising a terraced aerator for increasing the dissolved oxygen concentration of a liquid within the device. In an embodiment, the terraced aerator comprises a flowform.

This invention provides a terraced aerator comprising: one or more terraces; a means for suspending the terraced aerator all or partially above a liquid reservoir; and below a plant, seed, or a growth medium suspending the plant or seed; wherein: a liquid descending from the plant or seed or growth medium, through a gas comprising oxygen, to the first terrace; and the liquid descending from the first terrace through a gas comprising oxygen into the liquid reservoir; increases the dissolved oxygen content in the liquid or in the liquid reservoir, or both; and wherein each of the liquid descending steps produces a sound of less than about 57 decibels or wherein each of the liquid descending steps dampens the sound produced compared to the liquid descending to the liquid reservoir without contacting the terraced aerator.

This invention provides a terraced aerator comprising: two or more terraces; a means for suspending the first terrace above the second terrace; and a means for suspending the terraced aerator all or partially above a liquid reservoir; and below a plant, seed, or a growth medium suspending the plant or seed; wherein: a liquid descending from the plant or seed or growth medium, through a gas comprising oxygen, to the first terrace; and the liquid descending from the first terrace through a gas comprising oxygen to the second terrace; or the liquid descending from the second terrace through the gas into the liquid reservoir; or both from the first terrace through a gas comprising oxygen to the second terrace and the liquid descending from the second terrace through the gas into the liquid reservoir; increases the dissolved oxygen content in the liquid or in the liquid reservoir, or both; and wherein each of the liquid descending steps produces a sound of less than about 57 decibels or wherein each of the liquid descending steps dampens the sound produced compared to the liquid descending to the liquid reservoir without contacting the terraced aerator.

In an embodiment, the combined liquid descending steps produces a sound of less than about 57 decibels. In an embodiment, the terraces have one or more holes for the liquid to pass through.

In an embodiment, the one or more holes have diameters less than cross-sectional diameters of drops or streams of the descending liquid; or are less than 200 microns, less than about 200 microns, less than about 350 microns, less than about 500 microns, less than about 1000 microns, less than about 2000 microns, or less than about 5000 microns. In an embodiment, all of the liquid descending from the first terrace contacts the second terrace. In an embodiment, all of the liquid descending from the plant, seed, or growth medium contacts the first terrace. In an embodiment, the height distance between the first and second terraces is between about 0.5 inch and about 1 inch. In an embodiment, the terraces are capable of containing liquid or not containing liquid. In an embodiment, the second portion of liquid contacts the plant, seed, or growth medium and descends in drops into the remaining liquid, wherein each distance segment a drop falls between the first terrace and the second terrace or between the second terrace and the remaining liquid through the gas is the drop distance, wherein the device also comprises a means for decreasing or increasing the drop distance. In an embodiment, the terraced aerator is for a hydroponics device. In an embodiment, one or more of the terraces is a flowform. In an embodiment, all of the terraces are flowforms.

This invention provides a method for increasing the dissolved oxygen concentration in a liquid within a hydroponics device comprising: providing a hydroponics device containing a liquid to be delivered to a plant; a gas comprising oxygen above the liquid; a means for elevating a portion of the liquid in the gas above the remaining liquid; a means for delivering the portion of liquid into the gas; and a terraced aerator suspended in the gas above the liquid; elevating a portion of the liquid above the remaining liquid; delivering the portion of liquid into the gas; and allowing the portion of liquid to descend through the gas onto the terraced aerator and into the remaining portion of liquid; wherein the dissolved oxygen concentration in the liquid is increased. In an embodiment, the terraced aerator comprises a flowform.

This invention provides an aspirator for increasing the dissolved oxygen concentration in a liquid in a hydroponics system, the aspirator comprising a tube in which the liquid flows, wherein the tube comprises a gas inlet for receiving a gas comprising oxygen, whereby when the liquid flows through the tube the gas enters the tube and mixes with the liquid. In an embodiment, the aspirator is a downdraft venturi.

This invention provides a downdraft venturi for increasing the dissolved oxygen concentration in a liquid in a hydroponics system, the venturi comprising: a tube, the tube having an upper, first cross-sectional area and an area of transition to a lower, second, smaller cross-sectional area, the tube for descent of the liquid; and a gas inlet into the tube at about the area of transition; wherein descent of the portion of liquid through the tube draws a gas comprising oxygen in the gas inlet, whereby the gas mixes with the liquid and increases the dissolved oxygen concentration in the liquid.

In an embodiment, the upper portion of the tube described by the first cross-sectional diameter is about completely filled with the liquid. In an embodiment, the tube empties into a liquid reservoir containing a reservoir liquid. In an embodiment, the gas mixes with the liquid as the liquid contacts the surface of the reservoir liquid.

This invention provides a hydroponics device for growing a plant or germinating a seed, the device comprising a downdraft venturi for increasing the dissolved oxygen concentration of a liquid within the device. In an embodiment, the hydroponics device is enclosed.

In an embodiment, the hydroponics device comprises: a vessel for containing a liquid; a means for removably suspending the plant in a gas above the liquid; a conduit in fluid communication with the liquid and the gas; and a means for delivering a first portion and a second portion of the liquid through the conduit whereby the first portion of liquid falls through the gas into the remaining liquid in the vessel and the second portion of liquid contacts the plant, seed, or a growth medium contacting the plant or seed, and descends into the remaining liquid; whereby the root of the plant is permitted to grow in the gas and in the remaining liquid.

In an embodiment, the downdraft venturi comprises: a tube, the tube having an upper, first cross-sectional diameter and an area of transition to a lower, second, smaller cross-sectional diameter, the tube for descent of a liquid; and a gas inlet into the tube at about the area of transition; wherein descent of the portion of the liquid through the tube draws a gas comprising oxygen in the gas inlet, whereby the gas mixes with the liquid and increases the dissolved oxygen concentration in the liquid.

This invention provides a method for increasing the dissolved oxygen concentration in a liquid to be delivered to a plant, the method comprising: providing a liquid; providing a downdraft venturi in a gas comprising oxygen; delivering the liquid into the top of the downdraft venturi; and allowing the liquid to descend through the downdraft venturi wherein the gas enters into the downdraft venturi and mixes with the liquid. In an embodiment, the method further comprises: providing a hydroponics device for containing the liquid, the gas, and the downdraft venturi; and performing the delivering and the allowing steps within the hydroponics device.

This invention provides a net basket for supporting and delivering liquid to a plant, seed that will germinate into a plant, or growth medium for contacting the seed or plant, the basket comprising at least one channel having a vertical component for transporting liquid wherein the plant or seed grows and wherein a root of the plant and the liquid are allowed to exit through one or more holes in the net basket.

In an embodiment, the net basket also comprises at least one channel having a horizontal component for transporting liquid, wherein the channel having a horizontal component is in fluid contact with the channel having a vertical component. In an embodiment, the liquid is delivered in a horizontal or downward direction or both directions to the plant or a growth medium supported by the net basket. In an embodiment, the growth medium is a hydrophilic cellular substrate that expands when contacted by the liquid. In an embodiment, the liquid is directed to a side or bottom surface or both surfaces of the growth medium.

In an embodiment, the net basket further comprises a means for substantially preventing the liquid from contacting the uppermost surface of the growth medium. In an embodiment, the net basket also comprises a clog prevention means for preventing the growth medium from clogging one or both of the channels. In an embodiment, the clog prevention means is removable.

In an embodiment, the channel having a horizontal component comprises a proximal wall and a distal wall and the clog prevention means comprises a proximal wall of the channel having a horizontal component. In an embodiment, the proximal wall contacts and substantially sealingly contacts a distal wall of the channel having a horizontal component at about the top of the channel. In an embodiment, the channel having a vertical component comprises a proximal edge and wherein the clog prevention means comprises a proximal edge of the channel having a vertical component.

In an embodiment, the net basket further comprises a means for suspending the plant or seed in a hydroponics device. In an embodiment, the channel having a vertical component is a substantially vertical channel. In an embodiment, the net basket has four substantially vertical channels. In an embodiment, the channel having a horizontal component is a substantially horizontal channel. In an embodiment, having two or more substantially horizontal channels. In an embodiment, the horizontal channel is at about the bottom of the net basket. In an embodiment, a growth medium supported by the net basket rests upon at least a portion of the horizontal channel. In an embodiment, the horizontal channel retains a portion of the liquid. In an embodiment, the horizontal channel contacts the channel having a vertical component at about the top of the channel having a vertical component. In an embodiment, the basket has a perimeter wall having a proximal side wherein the channel having a horizontal component contacts the proximal side of a perimeter wall of the net basket. In an embodiment, the basket has two or more channels each having a vertical component equally spaced around the basket. In an embodiment, the basket comprises a liquid inlet at about the top of the channel having a vertical component. In an embodiment, the liquid inlet is at about the height of the transition region of the plant. In an embodiment, the liquid channel is U-shaped or L-shaped. In an embodiment, the liquid channel having a vertical component is an open channel and is open on the proximal side. In an embodiment, the liquid is delivered to the plant or seed through the channel having a vertical component. In an embodiment, the liquid is first transported through the channel having a vertical component, then through the channel having a horizontal component, and is then delivered to the growth medium. In an embodiment, the growth medium is a soil-less growth medium.

In an embodiment, the net basket comprises: four U-shaped horizontal channels at about the top of the net basket; four U-shaped vertical channels descending from the four horizontal channels wherein each vertical channel contacts two horizontal channels; a fifth L-shaped horizontal channel at about the bottom of the four vertical channels and at about the bottom of the net basket; and four liquid inlets at about the center of each of the four horizontal channels; wherein the liquid enters the net basket at the four liquid inlets, is transported along the four horizontal channels, is transported down the four vertical channels to the fifth horizontal channel, and exits the net basket through an opening in the bottom of the net basket. In an embodiment, the liquid contacts a growth medium supported by the net basket while being transported down the four vertical channels or while in the fifth horizontal channel, or both.

This invention provides a method for delivering liquid to a plant or seed that will germinate into a plant comprising: providing a net basket for supporting the plant or seed, the net basket comprising a liquid inlet and a channel having a vertical component for transporting the liquid; delivering a liquid to the liquid inlet; transporting the liquid through the liquid inlet to the channel having a vertical component; transporting the liquid through the channel having a vertical component; and contacting the plant or seed with the liquid; wherein the plant grows and wherein one or more roots of the plant and the liquid are allowed to exit through one or more holes in the net basket.

In an embodiment, the net basket also supports a growth medium contacting the plant or seed, wherein the liquid first contacts the growth medium and then contacts the plant or seed. In an embodiment, the method comprises: providing a net basket also comprising a channel having a horizontal component for transporting a liquid; and transporting the liquid to the channel having a horizontal component before or after transporting the liquid to the channel having a vertical component.

This invention provides a germination cap for increasing the likelihood of germination of a seed relative to an equivalent context without the cap, the cap comprising: a panel comprising at least a partially converging, diverging, refracting, or polarizing lens; and a means for supporting the panel between a photoradiation source and the seed; wherein the panel is at least partially permeable to photoradiation from the photoradiation source.

In an embodiment, the cap further comprises a means for increasing the temperature of the seed. In an embodiment, the cap further comprises a means for decreasing evaporation of a liquid contacting the seed. In an embodiment, the means for supporting the panel comprises one or more walls that contact the lens or panel and are able to contact a growth medium or growing surface near the seed. In an embodiment, the one or more walls form an airtight or gastight seal with the lens or panel and the growth medium or growing surface, thereby decreasing evaporation of a liquid contacting the seed or increasing the temperature of the seed or both. In an embodiment, the lens is selected from the group consisting of concave lenses, convex lenses, concave-concave lenses, plano-plano lenses, convex-convex lenses, fresnel lenses, plano-concave lenses, and plano-convex lenses. In an embodiment, the lens comprises a diffraction grating.

In an embodiment, the cap is for a hydroponics device. In an embodiment, the cap decreases evaporation of a liquid within the hydroponics device. In an embodiment, the cap is photopermeable. In an embodiment, the cap is translucent or transparent. In an embodiment, the cap is made from a material selected from the group consisting of glass, plastic, paper, and other photopermeable materials. In an embodiment, the photoradiation source is natural or artificial. In an embodiment, the panel is about flat or curved. In an embodiment, the panel is curved and a cross-section of the panel approximates an arc of a circle. In an embodiment, the cap is in the form of a covered cylindrical tube. In an embodiment, the cap creates about a greenhouse or terrarium environment.

In an embodiment, the means for supporting the panel supports the panel far enough away from the seed whereby the seed can germinate and grow for at least about 24 hours before the plant germinating from the seed contacts the germination cap. In an embodiment, the seed has a greater likelihood of germination with increased photoradiation and the lens is converging or wherein the seed has a greater likelihood of germination with decreased photoradiation and the lens is diverging. In an embodiment, the lens is converging and photoradiation produced by the photoradiation source is focused on the seed or wherein the lens is diverging and photoradiation produced by the photoradiation source is focused away from the seed.

This invention provides a set of germination caps for increasing the likelihood of germination of a plurality of seed types relative to an equivalent context without the set of caps, comprising two or more germination caps wherein a first germination cap comprises a converging lens and a second germination cap comprises a diverging lens.

This invention provides a method for increasing the likelihood of germination of a plurality of seed types relative to an equivalent context without the caps, the method comprising: providing a seed; providing a liquid and a means for contacting the seed with the liquid; providing a photoradiation source for delivering photoradiation to the seed; providing a germination cap: contacting the seed with the liquid; delivering the photoradiation at the seed; and converging or diverging the photoradiation towards or away from the seed; wherein the likelihood of germination of the seed is increased relative to delivering the photoradiation without converging or diverging.

This invention provides a set of germination caps for increasing the likelihood of germination of a plurality of seed types relative to an equivalent context without the set of caps comprising two or more germination caps wherein a first germination cap comprises: a first panel comprising at least a partially converging lens; and a means for supporting the first panel between a photoradiation source and the plurality of seed types; and a second germination cap comprising: a second panel comprising at least a partially diverging lens; and a means for supporting the second panel between a photoradiation source and the plurality of seed types; wherein the first and second panels are at least partially permeable to photoradiation from the photoradiation source. In an embodiment, the plurality of seed types comprises a lettuce seed and a cilantro seed wherein the first photoradiation converging cap is useful for increasing the likelihood of germination of the lettuce seed and wherein the second photoradiation diverging cap is useful for increasing the likelihood of germination of the cilantro seed. Plants differ in the amount of photoradiative light required for seed germination. Plant seeds that germinate better with light include: Godetia, Petunias, Snapdragons, Oriental Poppies, and English Daisies. Plant seeds that germinate better with less light include: Calendula, Nasturtiums, and other varieties of poppies. Light and darkness requirements of many seed types are known in the art (Bubel, Nancy, The New Seed Starters Handbook, pp 34-35).

This invention provides a method for increasing the likelihood of germination of a seed comprising: providing a seed; providing a liquid and a means for contacting the seed with the liquid; providing a photoradiation source for delivering photoradiation to the seed; providing a means for converging or diverging the photoradiation towards or away from the seed; contacting the seed with the liquid; and delivering the photoradiation to the seed comprising converging or diverging the photoradiation towards or away from the seed; wherein the likelihood of germination of the seed is increased relative to delivering the photoradiation without converging or diverging the photoradiation.

In an embodiment, the means for converging or diverging the photoradiation comprises covering the seed with a germination cap. In an embodiment, the germination cap comprises: a panel comprising at least a partially converging or diverging lens; and a means for supporting the panel between a photoradiation source and the seed; wherein the panel is at least partially permeable to photoradiation from the photoradiation source. In an embodiment, the method is performed using a hydroponics device.

This invention provides a method for increasing the likelihood of germination of a plurality of seed types, the method comprising: providing a plurality of seed types comprising a first seed and a second seed; providing a liquid and a means for contacting the first and second seeds with the liquid; providing a photoradiation source for delivering photoradiation to the first and second seeds; providing a means for converging or diverging the photoradiation towards or away from each the first and second seeds; contacting the first and second seeds with the liquid; delivering the photoradiation to the first seed comprising converging the photoradiation towards the first seed; and delivering the photoradiation to the second seed comprising diverging the photoradiation away from the second seed; wherein the likelihood of germination of the seed is increased relative to delivering the photoradiation without converging or diverging the photoradiation.

This invention provides a seed support medium, comprising: a seed-bearing substrate superposed upon a plant growth medium contained within a modular receptacle. In an embodiment, the growth medium is a hydrophilic cellular substrate. In an embodiment, the modular receptacle is a characteristic selected from the group consisting of: rigid, porous, and cup-shaped. In an embodiment, the seed-bearing substrate is a hydrophilic fiber or is plant starch. In an embodiment, the plant growth medium is soil-less. In an embodiment, the seed-bearing substrate is an adhesive. In an embodiment, the seed-bearing substrate comprises adjuvants. In an embodiment, the plant growth medium is a synthetic polymer or a sponge. In an embodiment, the seed-bearing substrate comprises two or more types of seeds. In an embodiment, the plant growth medium is rock wool. In an embodiment, the plant growth medium comprises adjuvants.

In an embodiment, the seed support medium comprises a seal. In an embodiment, the seal is at least partially opaque. In an embodiment, the seal is at least partially transparent. In an embodiment, the seal is at least partially translucent.

This invention provides a seed support medium comprising: a seed-bearing hydrophilic cellular polymer substrate contained within a modular rigid receptacle. In an embodiment, the growth medium is a synthetic polymer. In an embodiment, plant growth medium is sponge. In an embodiment, the plant growth medium is rock wool. In an embodiment, the plant growth medium further comprises adjuvant. In an embodiment, the plant growth medium further comprises a seal.

This invention provides a method for germinating a seed comprising: placing a seed supporting and growth medium comprising a seed-bearing substrate superposed upon a growth medium contained within a modular, rigid receptacle; delivering an aqueous liquid to the seed; and allowing the seed to germinate. In an embodiment, the seed supporting and germinating medium is placed in a hydroponics device and the aqueous liquid is delivered by turning on the hydroponic device thus allowing liquid nutrient to contact the supporting and germinating medium. In an embodiment, the hydroponics device is an aeroponics device. In an embodiment, the method also comprises delivering photoradiation to the seed before allowing the seed to germinate.

This invention provides a smart garden device for a hydroponics device, the hydroponics device having at least one characteristic or component, the smart garden device comprising: means for delivering electricity to the smart garden device; at least one timer; and means for determining, receiving, sending, or processing data regarding the status of the component or characteristic of the hydroponics device.

In an embodiment, the device also comprises a means for displaying the status of the component or characteristic. In an embodiment, the device also comprises a means for displaying the status of requirement to add nutrient or for displaying the status of requirement to add liquid or both. In an embodiment, the device also comprises a means for displaying the status of requirement to add liquid nutrient solution. In an embodiment, the device also comprises a timer for display of a requirement to add nutrient. In an embodiment, the timer has a two-week cycle.

In an embodiment, the hydroponics device also has a second component or characteristic, the smart garden device also comprising a means for determining, receiving, sending, or processing data regarding the status of the second component or characteristic of the hydroponics device or the smart garden device also comprising a means for displaying the status of the second component or characteristic or both. In an embodiment, the first and second components or characteristics are the same. In an embodiment, the first and second components or characteristics are different. In an embodiment, the component or characteristic is selected from the group consisting of: timers, timing cycles, photoradiation sources, pumps, need for nutrient, need for liquid within the device, humidity, root density, nutrient concentration, dissolved oxygen concentration, turbidity of liquid within the device, incident photoradiation, temperature, pH, and plant mass. In an embodiment, the liquid is water. In an embodiment, the liquid is liquid nutrient solution.

In an embodiment, the means for determining, receiving, sending, or processing data comprises a preprogrammed storage device. In an embodiment, the preprogrammed storage device is a circuit board. In an embodiment, the preprogrammed storage device is a computer chip. In an embodiment, the means for determining, receiving, sending, or processing data comprises a programmable storage device. In an embodiment, the programmable storage device is a circuit board. In an embodiment, the programmable storage device is a computer chip.

In an embodiment, the smart garden device comprises a means for determining, receiving, sending, or processing data regarding the status of two or more components or characteristics of the device and a means for displaying the status of two or more components or characteristics of the device.

In an embodiment, the smart garden device comprises a means for receiving data regarding the status of a photoradiation source, resetting a timer for the requirement to add nutrient, and selection of a timing cycle for a photoradiation source and/or a pump. In an embodiment, the smart garden device comprises a timer for a photoradiation source and a pump. In an embodiment, the smart garden device comprises a plurality of timing cycles for the timer. In an embodiment, the timing cycles are selected from the group consisting of: 24 hours on, 24 hours off, 20 hours on and 4 hours off, 18 hours on and 6 hours off, 16 hours on and 8 hours off, 14 hours on and 10 hours off, and 12 hours on and 12 hours off.

In an embodiment, the smart garden device further comprises a liquid level gauge and a means for detecting a signal from the liquid level gauge. In an embodiment, the means for detecting a signal from a liquid level gauge is a photocell. In an embodiment, the smart garden device also comprises a means for sending data to or receiving data from an external programmable storage device. In an embodiment, the external programmable storage device is accessed through the internet. This invention provides machine-readable storage devices, program storage devices, and programmable storage devices having data and methods for diagnosing physical conditions.

This invention also provides methods for using hydroponics devices and for growing plants and germinating seeds into plants using the smart garden devices of this invention.

In FIG. 13A, an embodiment of the present invention is comprised of three distinct superposed substrates for carrying and germinating a seed and supporting the resulting plant. The most superposed seed-bearing substrate 185 is comprised of paper material formed from a liquid pulp solution comprised of suitable fibers such as cellulose or cotton which upon drying provides a light-weight, stable, hydrophilic medium similar to paper. Because it is a liquid, the versatile pulp solution may be made to conform to any number of desired shapes, sizes or surfaces. Seeds 110 may be mixed into the pre-poured pulp solution, or they may be inserted more or less superficially onto the poured solution. Once the pulp solution dries, the seed is trapped within or upon the paper substrate. In one embodiment of the invention, the substrate is first poured into a shaped, modular mold, then imbedded with seeds and allowed to dry. This dried modular paper unit is further imbedded into a cellular urethane substrate as depicted in FIG. 13A.

In an alternative of this particular embodiment, a flat layer of pulp solution may be poured and then seeds are placed upon the wet substrate and made to adhere as the solution dries. The flat, dry paper layer may then be cut into modular units. In still another alternative of this particular embodiment, seeds are mixed into the pulp solution prior to pouring. The seed-bearing pulp solution may be poured into a layer, allowed to dry and then cut into modular paper units, or the seed-bearing pulp solution may be poured into molds and allowed to dry into modular paper units. In each of these alternatives, the paper medium is made first, and then superposed upon a cellular urethane polymer substrate consolidated with select aggregate product. In yet another alternative to this particular embodiment, the pulp solution could be poured directly into a concaved recess in the cellular urethane polymer substrate and then allowed to dry.

Another embodiment of the most superposed seed-bearing substrate comprises a sticky adhesive substance to which seeds will adhere and which itself will adhere to the intermediate hydrophilic cellular substrate.

The growth medium 109, which in FIGS. 13A-B is a hydrophilic cellular substrate, comprises the second superposed material. One suitable material is formed from a urethane pre-polymer reacted with an aqueous slurry of nutritive aggregate such as peat or bark, plus any number of desired adjuvants, fungicides, etc. In an embodiment of the present invention, the cellular urethane polymer substrate containing nutritive aggregate product, adjuvant, fungicide, etc., is formed directly within a shaped, modular receptacle 182 of coir, hemp or other suitable natural or synthetic material, which durable modular receptacle constitutes the third distinct and outermost substrate of the present invention. Or, in an alternative embodiment, the pre-shaped cellular urethane polymer substrate 109 may be pre-formed and inserted “dry” into the shaped, modular receptacle 182. The hydrophilic cellular substrate growth medium 109 may also be composed of natural sponge, or any other suitable polymer. It may also be composed of rock wool or horticultural foam which is a rigid hydrophilic cellular polymer.

In either alternative embodiment regarding the hydrophilic cellular substrate growth medium 109, the top surface of that substrate will bear a concave recess 184 suitable for holding the paper seed-bearing substrate 185. The paper 185 will be held within the concave recess 184 either by friction or by adhesion.

In an embodiment of present invention, the third, outermost substrate 182 consists of a shaped, modular receptacle comprised of durable, hydrophilic fibers such as coir, hemp or other suitable natural or synthetic material. This durable unit is shaped into a tapered cup whose specific design and size may vary according to the type of plant cultivated, the duration of the cultivation cycle and the specifications of the particular growing system used. Conceivable diameters of the unit range from about ¼ inch to about 4 inches or more. The outer rim of the durable cup-shaped unit is fashioned with an extra lip or ledge 186, which lip or ledge provides the stability necessary for supporting the entire plant grown in an aeroponic system. In general, the cup will taper inward, with the bottom of the cup having significantly smaller diameter than the lip. This taper provides easier transplanting and less root damage if the plant is transplanted to larger growing systems or into soil.

This unique modular seed support medium comprised of the three described substrates represents an improved seed-germination medium. The inventors have determined that this unique combination of substrates provides a distinct advantage for seed germination, especially in an aeroponic system, over any one of the substrates by itself. Each of the distinct substrates contributes uniquely and beneficially to seed germination, root growth and plant growth. The dry paper substrate 185 holds the seed 110 while controlling germination until a desired time when aqueous solution is applied to the paper 185 in order to dissolve the paper 185 and germinate the seed 110.

The hydrophilic cellular substrate growth medium 109 holds this seed-bearing pulp 185 while the seed 110 germinates. Most importantly, it provides a rooting substrate into which roots may attach and grow. This substrate further contains adjuvants that help to optimize plant growth. These adjuvants include nutrients such as calcium, phosphorous, and nitrogen and antifungals, anti-algals such as grapeseed extract, and beneficial bacteria, for example.

Furthermore, according to its design, the refined porosity of the cellular urethane 109 controls delivery of moisture or aqueous nutrient solution and air both to the seed and especially to newly-sprouted plant roots.

However, the inventors have discovered that the cellular urethane substrate alone does not possess sufficient mechanical integrity to support a plant for its entire life within an aeroponic system, nor is the cellular urethane substrate particularly well-suited for packaging, shipment, implantation and transplantation because of its insufficient mechanical integrity. The inventors have determined that the fibrous, durable outer cup-shaped substrate 182 provides the requisite rigidity, stability and durability to withstand packaging, shipment, implantation and transplantation, while protecting the more delicate nutritive cellular urethane substrate and the seed substrate it bears. Most essential for an aeroponic application, the durable, fibrous cup-shaped substrate can be designed into a shape that will hold a plant firmly in place in an aeroponic system throughout the life of the plant. If desired, the rigid, cup-shaped substrate 182 will maintain its shape and stability sufficiently to enable removal of the entire seed germination medium, along with a partially or fully-matured plant, from an aeroponic or hydroponic system.

Furthermore, this durable, fibrous, outer substrate 182 helps to control moisture by contacting with an aqueous nutrient solution, wicking that solution into the intermediate cellular urethane substrate, which itself wicks nutrient solution to a seed and then to young plant roots after germination. The coarseness of the fibers allows sufficient air to permeate the outer substrate and the intermediate cellular urethane substrate to aid in oxygenation of young plant roots. The coarse fibers also wick away excess moisture or allow air to evaporate excess moisture from the sponge substrate 109. It is conceivable that adequate moisture will reach the seed to permit healthy germination even if this durable outer cup-shaped receptacle is fashioned from a hydrophobic substance such as perforated plastic or a wire mesh. Coarse hydrophilic fibers provide the best substrate, however.

The seed-germination medium of the present invention is uniquely well suited for use in an aeroponic system. The modularity of the medium makes it ideally suited for implantation into and transplantation from the system. The durable modular unit of the present invention may be manufactured, packaged, stored for months and/or shipped to the consumer, who then simply has to unpackage the modular unit and insert it into a suitably sized hole in the surface of the aeroponic system. The consumer then needs merely to initiate the spray apparatus of the aeroponic system and the seed will germinate and grow without further attention. If the consumer wishes to utilize the aeroponic apparatus especially for seed germination, the durable, three-part medium of the present invention provides an ideal, transplantable seedling vessel. Finally, the modular unit is easily imbedded with any number of distinct varieties of seed, so that the entire unit may be conveniently labeled and identified. Certain nutrients may be absorbed into the cellular urethane layer or mixed into the pulp solution that optimizes the growth of a particular plant species.

In an alternative embodiment of the invention shown in FIG. 13B, only two distinct materials are used, namely a hydrophilic cellular substrate growth medium 109, which itself bears the seed 110, and the outer durable fibrous cup-shaped substrate 182. In this two-part embodiment of the invention, seeds 110 would be mixed into the aqueous slurry with which the urethane pre-polymer is reacted to form the cellular urethane 182. In this way the seeds become embedded into the sponge during its formation. Alternatively, the seed may be placed with some precision within subjacent layers of freshly formed cellular urethane. This second alternative is advantageous in that the placement and number of seeds within the sponge may be carefully controlled. This separate, two-part embodiment of the invention is advantageous over the three-part embodiment in that it eliminates one step in creating and inserting the paper substrate into the cellular urethane substrate, providing a simpler, more stable final product. In either the two-component or the three component seed-support medium described above, an additional seal 187 composed of a plastic, a metal foil or paper may be superposed upon the rim of the durable cup-shaped receptacle 182 in order to further benefit the seed 110. During storage and shipment, the seal 187 helps to preserve the mechanical integrity of the modular unit. After implantation of the seed 110 into a growing system, such as an aeroponics system, moisture will be applied to the inferior portion of the unit, namely the porous, cup-shaped receptacle 182. The seal 187 provides an additional advantage at this particular time in the growth cycle by trapping moisture within the unit and preventing evaporation until such time as the seed has effectively begun to germinate. It is advantageous, therefore, that the seal be comprised of a material that is impermeable to water. The seal 187 may then be conveniently removed to allow for the growth of the plant. Certain seeds germinate best in the dark, while others require light. Therefore, the seal 187 may be comprised of either an opaque substance or a transparent substance, or even a translucent substance, depending on the needs of a particular seed and plant species. The seal 187 also serves as a convenient label 172 for each modular unit, describing what type of seed is contained therein and optionally precise instructions for germination.

This invention provides devices for growing a plant or germinating a seed into a plant wherein the plant has one or more roots, the device comprising: a vessel for containing a liquid; a means for removably suspending the plant in a gas above the liquid; a conduit in fluid communication with the liquid and the gas; and a means for delivering a first portion and a second portion of the liquid through the conduit whereby the first portion of liquid falls through the gas into the remaining liquid in the vessel and the second portion of liquid contacts the plant and descends into the remaining liquid; whereby the one or more roots are permitted to grow in the gas and in the remaining liquid.

This invention provides methods for growing a plant or germinating a seed into a plant wherein the plant has a root, the method comprising: providing a vessel for containing a liquid; providing a means for removably suspending the plant in a gas above the liquid; providing a conduit in fluid communication with the liquid and the gas; and delivering a first portion and a second portion of the liquid through the conduit whereby the first portion of liquid falls through the gas into the remaining liquid in the vessel, and whereby the second portion of liquid contacts the plant and descends into the remaining liquid; whereby the root of the plant is permitted to grow in the gas and in the remaining liquid.

The devices can also include a means for delivering and the method can include delivering a third portion of the liquid through the conduit whereby the third portion of liquid falls through the gas, contacts the one or more roots, and contacts the remaining liquid. The second portion of liquid can contact the plant at about or below the height of the transition region of the plant. The devices of this invention can also include a terraced aerator for each plant to increase oxygenation while decreasing the decibel level of sounds produced by falling drops.

The methods and devices of this invention are useful for growing more than one plant. When more than one plant is grown, the device optionally includes a means for delivering and the method optionally includes delivering the second portion of liquid to each plant separately.

The first portion of liquid optionally only contacts the gas and the remaining liquid. Optionally the first portion of liquid is delivered substantially vertically downward.

Optionally the conduit has separate exits for the first and second portions of liquid. The means for delivering liquid can include a pump. Optionally the liquid and the one or more roots are completely contained in one vessel.

The first portion of liquid falling through the gas into the remaining liquid increases the dissolved oxygen content in the remaining portion of liquid, and the first portion of liquid failing into the remaining liquid, increases the negative ions within the device.

The first portion of liquid optionally falls in drops. Drops have diameters greater than about 200 microns, greater than about 350 microns, greater than about 500 microns, greater than about 1000 microns, greater than about 2000 microns, or greater than about 5000 microns.

This invention provides kits for growing a plant or germinating a seed into a plant comprising: a device for growing a plant or germinating a seed into a plant wherein the plant has one or more roots comprising: a vessel for containing a liquid; a means for removably suspending the plant in a gas above the liquid; a conduit in fluid communication with the liquid and the gas; and a means for delivering a first portion and a second portion of the liquid through the conduit whereby the first portion of liquid falls through the gas into the remaining liquid in the vessel and the second portion of liquid contacts the plant and descends into the remaining liquid; whereby the one or more roots are permitted to grow in the gas and in the remaining liquid; and instructions for using the device.

Optionally the first and second portions are delivered continuously. The methods of this invention optionally further comprise adding additional liquid, above pH 5.5, to the device.

This invention provides a method for delivering oxygen to a plant comprising: providing a plant with at least one root; providing a liquid capable of having oxygen dissolved therein; providing a gas comprising oxygen gas; providing a means for contacting and fluidly contacting the liquid with the gas whereby a portion of the oxygen gas dissolves in the liquid thereby forming oxygenated liquid; providing a means for elevating and elevating a portion of the oxygenated liquid above the remaining portion of oxygenated liquid; allowing the portion or oxygenated liquid to fall through the gas into the remaining portion of oxygenated liquid whereby more oxygen gas dissolves in the liquid thereby forming super-oxygenated liquid; and contacting the root with the oxygenated liquid or the super-oxygenated liquid; whereby oxygen is delivered to the plant.

Optionally the liquid falling through said gas into said remaining portion of oxygenated liquid increases the humidity level of said gas, and the method further comprises contacting said root with said humidity. Optionally the method further comprises contacting said root with said gas comprising oxygen. Optionally the method further comprises allowing said root to grow in said oxygenated or super-oxygenated liquid. Optionally the second portion of liquid cascades down terraces of a terraced aerator or terraced oxygenator.

This invention provides a reliable method for reminding a user to care for the growing plants. This invention provides a device having a means for alerting a user when to add water and a means for alerting the user when to add food (nutrients). This invention provides a device having a photoradiation source and a means for regulating the duration and frequency that photoradiation is delivered, and also a means for overriding the regulating means. This invention also provides a device for regulating the duration and frequency of a liquid delivery means.

The methods and devices of this invention are useful for quickly growing healthy productive plants. The devices of this invention include small, self-contained, portable devices for a home garden through large devices useful in the agricultural industry. The method and devices of this invention require no prior experience with growing plants, but also provide satisfying experiences and harvests for master gardeners. The methods and devices of this invention are useful for growing ornamental plants as well as plants for culinary use. The devices of this invention are useful for growing plants at all stages, including from seed through harvests, growing plants from seed for transplant, growing plants from seedlings, and growing cuttings. Reproductive and vegetative tissues including flowers, shoots, leaves, and roots can all be produced and harvested using the methods and devices of this invention. When using the methods and devices of this invention, the volume of the vessel is selected for the type and number of plants to be grown.

This invention provides methods, devices, and kits that are useful for growing plants hydroponically or with soil. This invention provides a device for growing a plant or germinating a seed into a plant wherein the plant has one or more roots, the device comprising: a vessel for containing a liquid; a means for removably suspending the plant in a gas above the liquid; a conduit in fluid communication with the liquid and the gas; and a means for delivering a first portion and a second portion of the liquid through the conduit whereby the first portion of liquid falls through the gas into the remaining liquid in the vessel and the second portion of liquid contacts the plant and descends into the remaining liquid; whereby the one or more roots are permitted to grow in the gas and in the remaining liquid. In an embodiment of this invention, the first portion of liquid falls in drops.

In an embodiment of this invention, the device also comprises a means for delivering a third portion of the liquid through the conduit whereby the third portion of liquid falls through the gas, contacts the one or more roots, and then contacts the remaining liquid. In an embodiment of this invention the third portion of liquid falls in drops.

In an embodiment of this invention, the plant opening removably suspends a plant growth medium or a plant support medium. In an embodiment of this invention, the first portion of liquid exits the conduit and does not contact a plant support medium, a plant growth medium, the plant, or a wall surface of the vessel before falling into the remaining liquid. In an embodiment of this invention, the first portion of liquid only contacts the gas and the remaining liquid. In embodiments of this invention, the first portion of liquid is delivered substantially vertically downward. In an embodiment of this invention, the first portion of liquid falling through the gas into the remaining liquid increases or maintains the dissolved oxygen content of the first and remaining portions of liquid. In an embodiment of this invention, the first portion of liquid falling into the remaining liquid increases the negative ions within the device.

In an embodiment of this invention, the second portion of liquid is delivered substantially horizontally. In an embodiment of this invention, the second portion of liquid contacts a plant support medium or a plant growth medium before contacting the plant. In an embodiment of this invention, the plant support medium or plant growth medium has a pH less than 8, less than about 7.9, less than about 7.5, or about 6.5. In an embodiment of this invention, the second portion of liquid contacts the plant at about or below the transition region of the plant. In an embodiment of this invention, the second portion of liquid contacts the one or more roots of the plant. In an embodiment of this invention, the second portion contacts the plant from three or more directions, each having a different horizontal direction component. In an embodiment of this invention, the second portion contacts the plant from essentially all horizontal directions, optionally by first contacting and flowing around an annular ring. In an embodiment of this invention, the device further comprises a flow-directing annular ring at about the plant opening.

In an embodiment of this invention, the means for delivering a first portion and a second portion of the liquid comprises a pump. In an embodiment of this invention, the first and second portions of liquid exit the conduit at an acceleration greater than 9.8 m/s² or at about 9.8 m/s² (gravity on earth). In an embodiment of this invention, the device also comprises a wicking means for delivering a fourth portion of liquid to the plant.

In an embodiment of this invention, the one or more roots are permitted to hang into the vessel wherein the vessel is not designed to have a structural element for the roots to lay on, other than the vessel bottom wall or any necessary components for other functions of the device. In an embodiment of this invention, the liquid and the one or more roots are completely contained in one covered vessel.

The devices of this invention are useful for growing more than one plant or seed. In an embodiment of this invention, the device also comprises a means for delivering the second portion of liquid to each plant separately.

In an embodiment of this invention, the gas comprises oxygen gas. In an embodiment of this invention, the liquid comprises water. In an embodiment of this invention, the liquid also comprises one or more plant nutrients. In an embodiment of this invention, the liquid comprises water and sufficient quantities of all the macronutrients and micronutrients necessary for optimal plant growth.

In an embodiment of this invention, the drops have diameters greater than about 200 microns, greater than about 350 microns, greater than about 500 microns, greater than about 1000 microns, greater than about 2000 microns, or greater than about 5000 microns.

In an embodiment of this invention, the conduit has separate exits for the first and second portions of liquid. In an embodiment of this invention, the conduit is a bifurcating conduit. In embodiments of this invention, the conduit is a closed conduit or an open conduit. In an embodiment of this invention, a closed conduit is used to allow the liquid to exit the conduit at an acceleration greater than 9.8 m/s².

In an embodiment of this invention, the cover comprises a removable lower and a removable upper cover. In an embodiment of this invention, the lower cover comprises a portion of the conduit. In an embodiment of this invention, the lower cover has one or more plant openings. In an embodiment of this invention, the upper cover has one or more plant openings horizontally aligned with the one or more plant openings of the lower cover.

This invention provides devices comprising a means for dampening the sound produced when the first, the second, or both portions of liquid descend, relative to without the means. In an embodiment, the sound is decreased or dampened from more than about 60 decibels to less than about 60 decibels, or less than about 57 decibels, as measured from outside an operating device of this invention, when the background sound level is about 52 decibels. In an embodiment, the means for dampening the sound comprises a terraced aerator. In an embodiment of this invention, the device also comprises a terraced aerator comprising: 1) a liquid directing means; 2) two or more terraces; and 3) a means for suspending the liquid directing means above a first terrace above a second terrace; and a means for suspending the terraced aerator in the gas above the liquid; wherein the second portion of liquid contacts the plant and descends to the liquid directing means, then descends from the liquid directing means to the first terrace, then descends from the first terrace to the second terrace, and then descends from the second terrace into the remaining liquid. In an embodiment, the second portion of liquid descends from the first terrace to the second terrace, from the second terrace to the remaining liquid, or both, in drops. In an embodiment the liquid descending in drops to the second terrace or into the remaining liquid produces a sound of less than about 57 decibels.

In an embodiment, the second portion of liquid contacts the plant and descends in drops into the remaining liquid, wherein each distance segment a drop falls through the gas is the drop distance, wherein the device also comprises a means for decreasing the drop distance.

This invention provides a kit for growing a plant or germinating a seed into a plant comprising: a device for growing a plant or germinating a seed into a plant wherein the plant has one or more roots comprising: a vessel for containing a liquid; a means for removably suspending the plant in a gas above the liquid; a conduit in fluid communication with the liquid and the gas; and a means for delivering a first portion and a second portion of the liquid through the conduit whereby the first portion of liquid falls through the gas into the remaining liquid in the vessel and the second portion of liquid contacts the plant and descends into the remaining liquid; whereby the one or more roots are permitted to grow in the gas and in the remaining liquid; and instructions for using the device. The kit optionally also comprises device assembly instructions. Optionally the device is already assembled.

This invention provides kits also comprising one or more terraced aerators. In an embodiment, one terraced aerator is provided for each plant.

In an embodiment of this invention the kit further comprises one or more of the components selected from the group consisting of: set of covers, seeds, plant supports, soil, lights, light stand, kit for using more than one device with one pump, a timer, one or more germination covers, a greenhouse lid, an external reservoir, a decorative outer vessel container, seeds, nutrients, means for detecting, providing, and/or modifying nutrients, photoradiation quantity and/or quality, temperature, fluid level, dissolved oxygen, pH of the liquid, means for detecting and quantitating unwanted organisms (e.g., anaerobic bacteria and algae), means for reporting results of various assays. Optionally, a device of this invention comprises a means for preventing overfilling the liquid. The means for assaying and/or modifying can include use of machine readable storage devices, program storage devices, and data sets regarding which plants are being grown and optimal nutrient concentration, temperatures, pH levels, etc.

This invention provides a method for growing a plant or germinating a seed into a plant wherein the plant has a root, the method comprising: providing a vessel for containing a liquid; providing a means for removably suspending the plant in a gas above the liquid; providing a conduit in fluid communication with the liquid and the gas; and providing a means for delivering and delivering a first portion and a second portion of the liquid through the conduit whereby the first portion of liquid falls through the gas into the remaining liquid in the vessel, and whereby the second portion of liquid contacts the plant and descends into the remaining liquid; whereby the root of the plant is permitted to grow in the gas and in the remaining liquid.

In an embodiment of this invention, the first and second portions are delivered continuously. In an embodiment of this invention, the method further comprises delivering a third portion of the liquid through the conduit whereby the third portion of liquid falls through the gas, contacts the one or more roots, and contacts the remaining liquid. In an embodiment of this invention, the method further comprises delivering a fourth portion of liquid to the plant by wicking the liquid to the plant.

In an embodiment of this invention, delivery is performed by pumping. In an embodiment of this invention, the second portion is delivered by pumping. In an embodiment of this invention, the pumping is performed while the plant is germinating and/or while the plant is less than two weeks old. In an embodiment of this invention, the delivery comprises wicking. In another embodiment, delivering comprises gravity flow.

In an embodiment of this invention, the method further comprises increasing the dissolved oxygen content of the first and remaining liquid when the first portion of liquid falls through the gas into the remaining liquid. In an embodiment of this invention, the method further comprises increasing the negative ions within the vessel when the first portion of liquid falls into the remaining liquid.

In an embodiment of this invention, the method further comprises removably suspending a plant growth medium or a plant support medium in each of the one or more plant openings.

In an embodiment of this invention, the means for removably suspending the plant comprises providing an upper cover for removably covering a lower cover, wherein the upper cover has one or more plant openings horizontally aligned with the one or more plant openings of the lower cover.

In an embodiment of this invention, the method is a hydroponic method. In an embodiment of this invention, the method further comprises providing plant growth components comprising nutrients, oxygen, carbon dioxide, and photoradiation and delivering the plant growth components to the plant.

In an embodiment of this invention, the method further comprises adding one or more nutrients to the liquid. In an embodiment of this invention, the adding is performed about once a week. In an embodiment of this invention, liquid is added less frequently than every 11 days. In an embodiment of this invention, liquid is added about once a month. In an embodiment of this invention, the added liquid is above pH 5.5, about pH 6.5, and/or below about pH 8.0.

This invention provides a method for delivering oxygen to a plant comprising: providing a plant with at least one root or a cutting that will develop a root; providing a liquid capable of having oxygen dissolved therein; providing a gas comprising oxygen gas; providing a means for contacting and fluidly contacting the liquid with the gas whereby a portion of the oxygen gas dissolves in the liquid thereby forming oxygenated liquid; providing a means for elevating and elevating a portion of the oxygenated liquid above the remaining oxygenated liquid; allowing the portion of oxygenated liquid to fall through the gas into the remaining oxygenated liquid whereby more oxygen gas dissolves in the liquid thereby forming super-oxygenated liquid; and contacting the root with the oxygenated liquid or the super-oxygenated liquid; whereby oxygen is delivered to the plant. In an embodiment of this invention, sufficient oxygen is delivered to the plant that the plant grows. In an embodiment of this invention, sufficient oxygen is delivered to the plant that the plant optimally grows.

Optionally the liquid falling through said gas into said remaining portion of oxygenated liquid increases the humidity level of said gas, and the method further comprises contacting said root with said humidity. Optionally the method further comprises contacting said root with said gas comprising oxygen. Optionally the method further comprises allowing said root to grow in said oxygenated or super-oxygenated liquid.

In an embodiment of this invention, the method further comprises providing fresh gas comprising oxygen gas. In an embodiment of this invention, the method further comprises contacting the plant at about or below the transition region of the plant with the oxygenated liquid. In an embodiment of this invention, the plant is contacted with the oxygenated liquid at an acceleration greater than about 9.8 m/s².

In an embodiment of this invention, contacting the root comprises oxygenated or super-oxygenated liquid falling onto the root. In an embodiment of this invention, the method further comprises repeating elevating a portion of the oxygenated liquid above the remaining portion of oxygenated liquid; allowing the portion or oxygenated liquid to fall through the gas into the remaining portion of oxygenated liquid whereby more oxygen gas dissolves in the liquid thereby forming super-oxygenated liquid; and contacting the root with the oxygenated liquid or the super-oxygenated liquid; with the super-oxygenated liquid. In an embodiment of this invention, the root grows in the oxygenated or super-oxygenated liquid whereby oxygen is delivered to the root.

In an embodiment of this invention, the liquid falling into the remaining liquid when the oxygenated liquid falls through the gas into the remaining oxygenated liquid whereby more oxygen gas dissolves in the liquid thereby forming super-oxygenated liquid; increases the humidity of the gas. In an embodiment of this invention, the humidity contacts the root and delivers oxygen to the root.

This invention provides methods wherein the second portion of liquid contacts the plant and descends to a liquid directing means, then descends from the liquid directing means to a first terrace, then descends from the first terrace to a second terrace, and then descends from the second terrace into the remaining liquid.

In an embodiment of this invention, the vessel and the cover form an enclosed chamber, except for the plant openings.

In an embodiment of this invention, oxygen is delivered to a plant in six ways: roots grow in the oxygen containing gas, water having dissolved oxygen is delivered to the plant near at or below the transition region, water having dissolved oxygen is dropped onto one or more roots of the plant, water drops and increases the humidity and the moisture containing dissolved oxygen contacts the roots, water drops and splashed the roots growing in the gas, and roots grow in the water having dissolved oxygen. In addition, water having dissolved oxygen is dropped through a gas comprising oxygen gas directly into the remaining water thereby increasing the dissolved oxygen concentration of the water.

In an embodiment of this invention, the walls of the vessel are not permeable to photoradiation and the suspending means removably covers the vessel. In an embodiment of this invention, the vessel and cover prevent unnecessary evaporation of water and entry of photoradiation and unwanted organisms. Some evaporation is desirable, as is known in the art (Christopher Hall and William D Hoff, (May 1, 2001) Water Transport in Brick, Stone and Concrete, Routledge mot E F & N Spon; 1st edition) to assist in wicking the liquid up to the plant and to oxygenate the liquid as it is wicking. The suspending means is able to hold one or more plants. The plants are suspended by any means known in the art including by suspending a plant support such as a frictionally engaged sponge in the opening by friction or by a hanging basket that is filled with soil or other growth medium. Alternatively, the plants can be propped up by a portion of the vessel. In an embodiment of this invention, the vessel and cover are made of an opaque, light-colored plastic (e.g., acrylonitrile butadiene styrene, Magnum™, Dow Chemical, Pevely, Mo., U.S.A.) that is impermeable to water, not permeable to photoradiation, and that absorbs little photoradiation. In an embodiment of this invention, the device is an enclosed chamber except for plant openings, which are large enough to allow for radial growth of the stem of each plant.

The maximum fill line for a device of this invention is low enough that drops can fall directly into the remaining liquid and high enough that the pump can continuously deliver liquid to the plant. Preferably the maximum fill line is also high enough or the vessel large enough that the liquid does not need to be replenished inconveniently often. During the slow phase of plant growth, liquid may only need to be replenished about every two weeks, but during periods of high growth, liquid may need to be replenished daily.

In an embodiment of this invention, the pump hangs from the cover instead of resting on the bottom wall of the vessel. In an embodiment of this invention, the device comprises a means for adjusting the amount of the first portion of liquid that falls directly into the remaining liquid.

Germination covers are covers that prevent substantial evaporation of liquid from the device. They are useful for temporarily covering portions of a liquid delivery means, such as a plant support to prevent evaporation through an opening in a cover. Germination covers are optionally permeable to liquid and to photoradiation. Evaporation of liquid during germination not only causes liquid loss and concentrates dissolved nutrients, but it also reduces the temperature at the location of evaporation, which can decrease germination. Seeds have optimal germination temperatures as is known in the art. Tomato seeds prefer warmer temperatures and lettuce seeds prefer cooler temperatures, for example. Germination covers are useful for both types of seeds, but covers that are transparent to photoradiation are preferred for germinating tomato seeds because the photoradiation results in warmer temperatures for the seed, whereas covers that are not transparent to photoradiation, less permeable to photoradiation, or reflective of photoradiation, are preferred for germinating lettuce seeds. The degree of liquid permeability and photoradiation permeability of germination covers is selected to control the temperature at germination. Germination covers are also useful to prevent evaporation from an opening that does not have a plant in it.

The methods and devices of this invention are useful for all plant growth stages from germination through multiple harvests. After use, the vessel and covers can be cleaned, optionally in a dishwasher, before reuse.

The devices and kits of this invention optionally also comprise a liquid inlet, a liquid outlet, one or more germination covers, a greenhouse lid, a decorative outer vessel container, seeds, different filters for different types of plants or for harvesting different plant tissues, replacement filters, a pump, tubing, nutrients, means for detecting, providing, and/or modifying nutrients, photoradiation quantity and/or quality, temperature, fluid level, dissolved oxygen, pH of the liquid, means for detecting and quantitating unwanted organisms (e.g., bacteria and algae), means for reporting results of various assays. Optionally, a device of this invention comprises a means for preventing overfilling the liquid. The means for assaying and/or modifying can include use of machine readable storage devices, program storage devices, and data sets regarding which plants are being grown and optimal nutrient concentration, temperatures, pH levels etc.

In an embodiment of this invention, seeds are germinated on a removable plant support in a germination device, which can be a device of this invention, and after germination, the plant support and germinated seeds can be removed and placed in a second device, such as a device of this invention, for further growth. Optionally the second device comprises a vessel of a different size.

This invention provides a terraced aerator comprising a liquid directing means, two or more terraces, and a means for suspending the liquid directing means above a first terrace above a second terrace and above a liquid reservoir, wherein a liquid descending from the liquid directing means to the first terrace, the liquid descending from the first terrace through a gas comprising oxygen to the second terrace, and the liquid descending from the second terrace through the gas into the liquid reservoir, increases the dissolved oxygen content in the liquid and in the liquid reservoir, and wherein each of the liquid descending steps produces a sound below about 57 decibels, as measured from outside an operating device of this invention, wherein the background sound is about 52 decibels. In an embodiment, all of the liquid descending from the first terrace contacts the second terrace. In an embodiment, the terraced aerator comprises round, horizontal terraces that increase in diameter as the height above the surface of the liquid reservoir. In an embodiment, the height distance between the first and second terraces and between the second terrace and the surface of the liquid reservoir is between about 0.5 inch and about 1 inch.

In an embodiment, one or more of the terraces into which liquid drops contains liquid, e.g., is a concave shape or has side walls, wherein liquid falling in drops to the terrace falls into the liquid, thereby increasing the dissolved oxygen content of the liquid.

This invention provides devices, methods, and kits wherein liquid becomes aerated and oxygenated as it cascades down, onto one or more terraces. Preferably the liquid at least partially falls in drops onto each terrace and falls into liquid on or in each terrace, which, although applicants do not wish to be bound by any particular theory, applicant believes more greatly increases oxygenation of the liquid. The terraces decrease the drop distance, the distance a drop falls uninterrupted, e.g., by contact with anything but the gas, into another portion of liquid, which, although applicants do not wish to be bound by any particular theory, applicants believe decreases the decibel level of the sounds produced by the drop contacting the other portion of liquid. In an embodiment, the sound produced by the dropping liquid, in an operating device of this invention, is of a decibel level below about 60, 59, 58, 57, or 56 decibels relative to in an equivalent device without terraces, in background noise of about 52 decibels.

In an embodiment of this invention, the terraced aerator fluidly contacts a conduit exit and a second portion of liquid. In an embodiment, the terraced aerator also comprises a means for contacting the directing means to the cover, conduit exit, growth medium, and/or plant growth support, whereby all of the liquid that is not utilized by the plant descends down the terraced aerator. In an embodiment, the terraced aerator is removable, and is removed when a higher decibel sound, e.g., falling rain, waterfall, or fountain, is preferred by the user.

In an embodiment, all of the liquid not utilized by the plant contacts the directing means and streams, i.e., is always in liquid contact with and does not fall in drops, to the first terrace. The liquid in contact with the first terrace descends to the second terrace, optionally falls in drops or streams or a combination thereof. In an embodiment, the liquid then descends to each successive terrace in drops, streams, or combination thereof. The shapes and surfaces of the directing means, terraces, and means for suspending the directing means and terraces, and the drop distance are selected to achieve a desired level of oxygenation and a desired decibel level.

When making or selecting a device of this invention, the size of the vessel, number and size of the plant openings, the conduit configuration, etc. is selected to be appropriate for the types, expected sizes, and number of plants to be grown in the device.

In an embodiment, the ratio for volume of reservoir to the volume of the vessel is less than about 1:1 or less than 1:1. In an embodiment, the volume of the reservoir to the volume of the growth medium is greater than about 4:1 or about 6:1.

The devices of this invention are optionally free-standing or capable of being suspended.

In an embodiment of this invention, a liquid contacting a terrace of a terraced aerator falls directly to another terrace or a liquid reservoir and does not contact a plant. In an embodiment none of the terraces are utilized for supporting a plant. In an embodiment, the liquid flows through a hole in a terrace.

The net baskets, terraced aerators, downdraft venturi, and aspirators, soil-less seed supports, germination caps, smart garden devices, and methods of this invention are useful with plant growing systems and devices known in the art and as yet to be invented, in addition to hydroponics systems and devices.

In an embodiment, the pump delivery rate and configuration of the inside of the cover are selected to deliver about 3 ounces of liquid to each plant or seed per hour. In an embodiment of this invention, about 2 gallons of liquid are delivered to all the plant openings per hour. In an embodiment, about the ratio of the first portion of liquid to the second portion of liquid is between about 1:10 to about 10:1, or about 1:2.

This invention provides a device for growing a plant or germinating a seed into a plant, wherein said plant may have one or more roots, said device comprising: a vessel for containing a liquid; a means for removably suspending said plant in a gas above said liquid; a means for elevating a first portion of said liquid above the remaining liquid in said vessel and into said gas wherein said first portion of liquid falls through said gas into said remaining liquid or a means for contacting a second portion of said liquid with said plant, seed, or a growth medium contacting said seed or plant and allowing said first or second portion of liquid to return to the remaining liquid; whereby said one or more roots are permitted to grow in said gas and in said remaining liquid.

In an embodiment of this invention, the liquid falls through the gas in a direction having a vertical and a non-zero horizontal component.

In an embodiment of this invention, the first portion of liquid is prevented from contacting any of the plants growing within the device.

In an embodiment, the liquid exit holes have a diameter less than about 1 mm. In an embodiment, the liquid is delivered at about the height of the transition region and not substantially below the transition region height. In an embodiment, no liquid is uniformly sprinkled within the vessel.

In an embodiment, there is nothing preventing or decreasing the likelihood that all the roots of a plant grow into the reservoir.

The hydroponics devices of this invention optionally comprise a means for evacuating liquid within the device by means of a pump.

In an embodiment, a net basket within a hydroponics device of this invention is configured to not contact the liquid reservoir but only the gas within the device. The net baskets of this invention have one or more holes to allow a shoot and a root of a plant to grow out.

The devices and compositions of this invention are useful for growing one or more plants, germinating one or more seeds into plants, growing one or more bulbs into plants, growing one or more tubers into plants, growing one or more runners into plants, and/or rooting one or more cuttings into plants.

In an embodiment of this invention, the first and second portions of liquid are delivered simultaneously. In an embodiment of this invention, the means for delivering liquid and the means for delivering photoradiation are scheduled to operate simultaneously.

When making or selecting a net basket of this invention, the channel locations and shapes are selected to prevent a contained and supported wet growth medium from completely clogging any of the channels. When using a hydroponics device or net basket of this invention, a growth medium is selected for the plant that is to be grown and the delivery schedule of the liquid. In an embodiment of this invention, the growth medium is not soil-less and comprises soil. In an embodiment, the growth medium includes a variety of materials useful for growing plants. In an embodiment, plant nutrients are in the growing medium.

The methods and devices provided by this invention are useful with and without soil. The methods are easy to follow and the devices are easy to use. Most plants, including universally believed to be difficult growers such as orchids can be grown in the devices of this invention. The devices of this invention form enclosed chambers for root nourishment and growth. The devices are self-contained and provide water, photoradiation, and plant nutrients with little care and maintenance by a user. Optionally means are provided for alerting a user to add water, liquid, and/or plant nutrients. The devices optionally include photoradiation sources, and a means for regulating the frequency and duration of photoradiation delivery.

The devices of this invention are useful for growing plants from seed through harvest and through senescence or death. The devices of this invention are useful for growing transplants, cuttings, somatic embryos, tubers, and runners.

The devices of this invention can be used with plant nutrients that also contain human nutrients, making the edible plants grown in the devices of this invention more nutritious for humans consuming the plants.

Optionally reflective material is installed inside the artificial photoradiation hood of the devices of this invention. Optionally, the hydroponics device also includes a funneling apparatus for adding liquid into the device.

Optionally the hydroponics devices of this invention also include a battery to maintain the functioning of the timer(s) during short intervals in which electricity is not supplied, such as during power outages or during moving the device to a different location. Optionally an external electric cord connects the base to the photoradiation hood. The cord can be unplugged and an extension cord added to suspend the photoradiation hood at a higher elevation than permitted by the arm.

In an embodiment of this invention, the devices are made by injection molding ABS. The hydroponics device of this invention can be made from any material that is firm enough to hold liquid. The material used to make the cover and vessel are preferably sufficiently impermeable to photoradiation to prevent photoradiation from entering inside. The material for enclosing the water level gauge is preferably permeable to photoradiation, allowing the gauge to be visible. The materials that contact the plants or the liquid should not substantially reduce plant health or impede growth. Materials useful in the practice of this invention include, for example, glasses, plastics, and metals. Useful plastics include, for example, acrylonitrile butadiene styrene, polyethylene terepthalate glycol, polystyrene, polycarbonate, recycled, recyclable, photodegradable, and biodegradable plastics. Useful degradable plastics do not degrade during use of the device. Biodegradable plastic materials are particularly useful for terraced aerators and net baskets which may be transplanted with plants.

In an embodiment, the smart garden includes a means for communicating with an external programmable storage device directly and/or through the internet.

The devices for growing plants, terraced oxygenators; aspirators, downdraft venturis, net baskets germination caps, sets of germination caps seed-bearing support media and smart garden devices of this invention are useful alone and in combination, in the practice of this invention.

Downdraft venturi characteristics described herein are useful with aspirators of this invention. Characteristics of specific hydroponics devices described herein are useful with additional hydroponics devices of this invention. Characteristics of specific terraced aerators and oxygenators described herein are useful with additional terraced aerators and oxygenators of this invention. Characteristics of specific net baskets described herein are useful with additional modular receptacles of this invention.

In an embodiment, a seed-support medium also comprises a germination cap.

In an embodiment, an enclosure for the descending first portion of liquid is a structure that physically encloses the descending liquid from the point of falling to the reservoir liquid, thereby creating a sound barrier and maximizing the drop distance of the falling liquid by preventing materials such as roots from intersecting the falling path of the liquid.

This invention provides sets of seed support media comprising: a first seed-bearing hydrophilic cellular polymer substrate contained within a first modular rigid receptacle and a second seed-bearing hydrophilic cellular polymer substrate contained within a second modular rigid receptacle wherein said first seed is of a different variety or species than said second seed.

Hydroponics nutrients known in the art are useful in the practice of this invention, including liquid nutrient, powder nutrient, one-part, two-part, and three-part nutrient. Hydroponics additive are also useful in the practice of this invention. Additives can be added through the nutrient inlet or the door.

In an embodiment of this invention, a germination cap increases the likelihood of germination from about 1% to about 90%, from about 5% to about 50%, or from about 10% to about 25% relative to an equivalent context without the germination cap.

Adhesives and substrates useful in the practice of this invention do not substantially interfere with seed germination or plant growth.

External liquid reservoirs are useful with the devices and methods of this invention.

### EXAMPLE 1

A hydroponics device of this invention, including terraced aerators and net baskets, as shown in FIGS. 1A-D was made. White, smooth on two sides, extruded, utility grade with virgin cap, acrylonitrile butadiene styrene (ABS) plastic was purchased from Port Plastics (Denver, Colo., USA) and Professional Plastics (Denver, Colo., USA) which were manufactured by Spartech Plastics (St. Louis, Mo., USA) or Primex Plastics Corporation (Richmond, Ind., USA). This plastic was used for the vessel, cover, base, photoradiation hood, terraced aerators, venturi, net baskets, and support stand for the cover. The plastic for the liquid level gauge float window was polyethylene terephtalate glycol (PETG). Vinyl labels were used for the smart garden panel. Circuit boards for the smart garden were purchased from Digi-Key (Thief River Falls, Minn., USA). The processor for the circuit boards was purchased from National Semiconductor (Santa Clara, Calif., USA). The transformer, 12 V DC, 300 mA, was purchased from K-Mark Industrial Ltd (Hong Kong, China). Electric wires, 2-wire, AC, 16 gauge, were purchased from Home Depot (Atlanta, Ga., USA). Electric contacts between the base and device were purchased from Littlefuse (Des Plaines, Ill., USA). The reflective material for inside the photoradiation hood was purchased from (McMaster-Carr, Elmhurst, Ill., USA). The pump was a ViaAqua V880, AC 110-120V, 60 Hz, 3 W from Discount Pumps (Nipomo, Calif., USA). The pump was connected to the cover with polyvinyl chloride (PVC) tubing. The photoradiation bulbs were Marathon, Red or Daylight, 25 W, compact fluorescent bulbs purchased from Phillips Lighting Company (Somerset, N.J., USA). The transformer sends 3V to the control panel and 110V to the photoradiation apparatus. The device also contains a magnetic read switch for communicating data regarding the liquid level to the smart garden. The device was utilized to germinate dwarf tomato seeds using a soil-less growth medium from Grow-Tech, Inc. (Boothbay, Me., USA). The tomato plants were grown to maturity and cherry tomatoes were harvested.

The device used for Example 1 was configured to hold about a gallon of liquid, and to allow about 12 cups of the liquid to be available to the plants before the pump runs dry. The vessel is shown in FIG. 4B. The liquid level gauge (labeled in FIG. 1B) and the nutrient basket are shown in the vessel. The cover is shown in FIG. 4A. Except for at the plant openings, the cover prevents photoradiation from entering the vessel, when on the vessel. The cover is supported by the cover stand which surrounds the pump. Inside the stand, the pump is connected via a tube to the conduits running inside the cover. The cover is made from a lower and an upper cover that snap together to form the conduits. The cover also has a door which allows a user to view the roots while the plant grows and to add liquid, optionally water or nutrient liquid, to the vessel. The cover has a nutrient inlet cover, a door that can be opened to add plant nutrients to the liquid within the device. The nutrient inlet cover has been configured to be directly over the nutrient basket. Net baskets snugly fit into the plant openings. The net baskets can be supplied separately or with a growth medium, seeds, seed adhesive, a label, and/or a seal. The outermost rim of the net basket rests on the cover. When the device is filled with one gallon of water, there is a gas space beneath the net basket, above the gallon of liquid. Before the device is turned on, the gallon of liquid does not contact the net baskets. The cover also has a means for connecting to one or more terraced aerators, optionally to suspend a terraced aerators directly underneath each opening. The terraces are configured to be at heights that are about never submerged in the reservoir liquid and about always in the gas or that fluctuate between being submerged (i.e., below the height of the surface of the liquid reservoir) and being completely in the gas and partially submerged and partially in the gas. The terraced aerator is also configured to not interfere with the net basket or seed support medium (the net basket, the growth medium, and the one or more seeds). The conduits inside the cover have exits at the plant openings and also have one or more exits that are not at a plant opening. In this embodiment, there is one exit that is not at a plant opening. This exit is at about the top of an aspirator that is a downdraft venturi. The downdraft venturi empties into the liquid reservoir near the pump inlet. The nutrient basket has been placed to also be near the pump inlet to facilitate mixing of the nutrient with the liquid within the device. The vessel is designed to be free-standing or to rest in the base. The base connects to an adjustable arm which supports a photoradiation hood. The photoradiation hood can house bulbs which provide sufficient quantity and quality of photoradiation for growing plants. The base has a smart garden device for regulating the on/off cycles of the pump and the photoradiation apparatus, and for signaling when the device needs liquid and/or nutrient. Germination caps can be used that fit over the plant openings and the seed support media and that direct photoradiation from the photoradiation apparatus towards or away from the seeds underneath.

### EXAMPLE 2

The device in FIGS. 1A-D was used to germinate and grow tomatoes. A first seed support medium containing a first variety of dwarf tomato seeds (three seeds) was placed in a plant opening in the cover shown in FIG. 4A. A second seed support medium containing a second different variety of dwarf tomato seeds (three seeds) was placed in a second plant opening in the cover. The seed-support media were placed in non-adjacent openings. The seed support media were inserted with a twisting motion, to line up the liquid inlets with the exits in the conduit. The empty openings were covered with photoradiation impermeable covers. Terraced aerators were not used. Germination caps were not used.

The cover was placed on the vessel shown in FIG. 4B. The covered vessel was placed in a photoradiation stand shown in FIGS. 9A-D and arranged on a kitchen counter, in ordinary air. Electrical contacts connected the vessel, cover, and photoradiation apparatus. The photoradiation apparatus contained a smart garden device and a transformer. The photoradiation hood was set at the lowest setting, closest to the cover. The door was opened and normal tap water, not softened or well water that had not been subsequently filtered, was added until the water level indicator read full. The device was plugged in to a regular electrical outlet. The time of day or night at which the device was plugged in was the start time for the photoradiation on portion of a twenty-four hour cycle, e.g. if the device was plugged in at 6 AM, the photoradiation would have been delivered each day, beginning at 6 AM. The nutrient inlet cover was lifted and plant nutrient (FloraNova, General Hydroponics, Sebastopol, Calif., USA) was added, in the amount recommended by the manufacturer for one gallon of water. The plant nutrient was diluted in the liquid reservoir. The Add Nutrients reset button on the smart garden was pressed to reset the Add Nutrient timer. The exact type of nutrient added was changed as the plants grew to match the correct stage of growth, e.g. Grow or Flower/Bloom formula.

When electricity was supplied to the device, the liquid in the device entered the pump, was pumped up to the cover through a tube into the conduits within the cover. A first portion of liquid exited through an exit for a first portion and fell through the venturi, pulling in air, which contained oxygen, into the gas inlets in the venturi. The first portion of liquid and the air mixed and fell into the reservoir liquid remaining in the vessel, thereby increasing the concentration of dissolved oxygen in the liquid. A second portion of liquid exited the conduit at exits for the second portion of liquid at the plant openings. At the openings with no seed-bearing media, the second portion of liquid fell into the reservoir liquid. At the openings with seed-bearing media, the second portion of liquid then entered the net baskets at the liquid inlets, flowed along horizontal channels, down vertical channels, and into a horizontal channel in which the growth medium rested. The dry, shrunken growth medium absorbed some of the liquid and delivered it to the seed. The rest of the second portion of liquid fell off the seed support medium and fell in drops or streams through the gas into the reservoir liquid. After the growth media were moistened, liquid delivered to the modular receptacles, the net baskets, entered at the liquid inlets, flowed into the horizontal channels, and generally continued along the same flow pathway as when the growth medium was dry, however the liquid may have also contacted the expanded wet growth medium in any of the channels, but the structure of the net basket prevented the growth medium from clogging any of the channels completely.

The timing cycle selection button was pressed until “tomatoes” was lit up. Photoradiation and liquid nutrient were delivered for sixteen hours and not for eight hours, to make a twenty-four hour cycle. After several days, the tomato seeds germinated. In two weeks, the Add Nutrients light flashed. The same amount of nutrient was added through the nutrient inlet and the Add Nutrients Reset button was pushed. By this time, roots of the plants grew into the air above the reservoir liquid and into the liquid. Drops and streams of liquid ran down the roots into the reservoir. During the third week, the Add Water light flashed and more water was added through the door. Water was added until the water level indicator read full. At week four, the Add Nutrients light flashed again, and the same amount of nutrients was added. The Add Water light was now flashing more often, and water was added more often. During the second month, flowers grew on the tomato plants and tomatoes formed. Photoradiation and liquid continued to be delivered for sixteen hours of each twenty-four hour cycle.

During one evening, at about 7 pm, the photoradiation override button was pushed. Photoradiation was no longer delivered for the rest of the cycle, but liquid was delivered as usual, until 10 PM, when both would normally shut off if the device was first plugged in at 6 AM. The next morning, at 6 AM, both photoradiation and liquid was begun to be delivered, as usual.

If the user had decided to clean the device, the user would have pulled the device up out of the base resulting in electricity no longer being delivered to the pump, and therefore no more liquid being delivered to the plants. The device was set down by the kitchen sink. The cover, including the plants, was lifted off of the vessel and set on the counter. The liquid in the vessel was poured down the drain or on landscape plants outside. Fresh tap water was added to the vessel until the water level indicator read full. The cover and the plants were placed back on the vessel, while care was taken to ensure all roots were inside. The device was placed back in the base. Nutrient was added, and the Add Nutrient Reset button was pushed.

During the fourth month, tomatoes were harvested. Water and nutrient were added on this schedule for several more months. When the tomatoes stopped producing fruit, the device was disassembled and cleaned, and ready to be used to grow more plants.

### EXAMPLE 3

The device in FIGS. 1A-D is used to germinate and grow lettuce and cilantro. Four seed support media, each containing four seeds of one of four varieties of lettuce are placed in the back openings. Three seed support media, each containing four seeds of cilantro, are placed in the front three openings. Germination caps are used. Converging germination caps are used for the lettuce and diverging germination caps are used for the cilantro. An equivalent second device is set up without the germination caps. Water and nutrient are added to the devices and they are plugged in. A third device is set up with the germination caps in switched positions, so that the diverging caps are on the lettuce and the converging caps are on the cilantro. In the first device, about 100% of the seeds germinated. In the second device, about 75% of the seeds germinated. In the third device, about 50% of the seeds germinated.

### EXAMPLE 4

The device in FIGS. 1A-D is used to germinate and grow herbs, including: two varieties of basil, cilantro, dill, marjoram, parsley, and chives. During the second month, herbs are harvested and used in culinary recipes.

### EXAMPLE 5

The device in FIGS. 1A-D is used to germinate and grow flowers. Terraced aerators are used to dampen the sound produced by the falling drops and stream and to better oxygenate the liquid. Converging germination caps are used with Godetia, Snapdragons, and English Daisies. Diverging germination caps are used with Calendula and Nasturtiums.

Although this invention has been described with respect to specific embodiments, it is not intended to be limited thereto, and various modifications which will become apparent to the person of ordinary skill in the art are intended to fall within the scope of the invention as described herein, taken in conjunction with the accompanying drawings and the appended claims.

All references cited are incorporated herein by reference to the extent that they are not inconsistent with the disclosure herein.

1. (canceled)

2-3. (canceled)

4-13. (canceled)

14. (canceled)

15-18. (canceled)

19-29. (canceled)

30. (canceled)

31. (canceled)

32. (canceled)

33. (canceled)

34. (canceled)

35-53. (canceled)

54. (canceled)

55-63. (canceled)

64. (canceled)

65-210. (canceled)

211. (canceled)

212-215. (canceled)

216. A germination cap for increasing the likelihood of germination of a seed relative to an equivalent context without said cap, said cap comprising:
a) a panel comprising at least a partially converging, diverging, refracting, or polarizing lens; and
b) a means for supporting said panel between a photoradiation source and said seed;
wherein said panel is at least partially permeable to photoradiation from said photoradiation source.

217. The germination cap of claim 216 wherein said means for supporting said panel comprises one or more walls that contact the lens and are able to contact a growth medium or growing surface near said seed.

218. The germination cap of claim 217 wherein said one or more walls form an airtight seal with said lens and said growth medium or growing surface, thereby decreasing evaporation of a liquid contacting said seed or increasing the temperature of said seed or both.

219. The germination cap of claim 216 wherein said lens is selected from the group consisting of concave lenses, convex lenses, fresnel lenses, concave-concave lenses, plano-plano lenses, convex-convex lenses, plano-concave lenses, and plano-convex lenses.

220. The germination cap of claim 216 for a hydroponics device.

221. The germination cap of claim 220 wherein said cap decreases evaporation of a liquid within said hydroponics device.

222. The germination cap of claim 216 wherein said cap is translucent or transparent.

223. The germination cap of claim 216 wherein said cap is made from a material selected from the group consisting of glass, plastic, paper, and other photopermeable materials.

224. The germination cap of claim 216 wherein said panel is about flat or curved.

225. The germination cap of claim 224 wherein said panel is curved and a cross-section of said panel approximates an arc of a circle.

226. The germination cap of claim 216 wherein said cap creates about a greenhouse or terrarium environment.

227. The germination cap of claim 216 wherein said means for supporting said panel supports said panel far enough away from said seed whereby said seed can germinate and grow for at least about 24 hours before the plant germinating from said seed contacts said germination cap.

228. The germination cap of claim 216 wherein said seed has a greater likelihood of germination with increased photoradiation and said lens is converging or wherein said seed has a greater likelihood of germination with decreased photoradiation and said lens is diverging.

229. The germination cap of claim 216 wherein said lens is converging and photoradiation produced by said photoradiation source is focused on said seed or wherein said lens is diverging and photoradiation produced by said photoradiation source is focused away from said seed.

230. A set of germination caps for increasing the likelihood of germination of a plurality of seed types relative to an equivalent context without said set of caps comprising two or more germination caps of claim 216 wherein a first germination cap comprises a converging lens and wherein a second germination cap comprises a diverging lens.

231. A method for increasing the likelihood of germination of a seed comprising:
a) providing a seed;
b) providing a liquid and a means for contacting said seed with said liquid;
c) providing a photoradiation source for delivering photoradiation to said seed;
d) providing a germination cap of claim 216;
e) contacting said seed with said liquid;
f) delivering said photoradiation initially at said seed; and
g) converging or diverging said photoradiation towards or away from said seed;
wherein said likelihood of germination of said seed is increased relative to delivering said photoradiation without converging or diverging.

232. A set of germination caps for increasing the likelihood of germination of a plurality of seed types relative to an equivalent context without said set of caps comprising two or more germination caps wherein a first germination cap comprises:
a) a first panel comprising at least a partially converging lens; and
b) a means for supporting said first panel between a photoradiation source and said plurality of seed types; and
a second germination cap comprising:
1) a second panel comprising at least a partially diverging lens; and
2) a means for supporting said second panel between a photoradiation source and said plurality of seed types;
wherein said first and second panels are at least partially permeable to photoradiation from said photoradiation source.

233. A method for increasing the likelihood of germination of a seed comprising:
a) providing a seed;
b) providing a liquid and a means for contacting said seed with said liquid;
c) providing a photoradiation source for delivering photoradiation to said seed;
d) providing a means for converging or diverging said photoradiation towards or away from said seed;
e) contacting said seed with said liquid; and
f) delivering said photoradiation to said seed comprising converging or diverging said photoradiation towards or away from said seed;
wherein said likelihood of germination of said seed is increased relative to delivering said photoradiation without converging or diverging said photoradiation.

234. The method of claim 233 wherein said means for converging or diverging said photoradiation comprises covering said seed with a germination cap.

235. A method for increasing the likelihood of germination of a plurality of seed types, said method comprising:
a) providing a plurality of seed types comprising a first seed and a second seed;
b) providing a liquid and a means for contacting said first and second seeds with said liquid;
c) providing a photoradiation source for delivering photoradiation to said first and second seeds;
d) providing a means for converging or diverging said photoradiation towards or away from each said first and second seeds;
e) contacting said first and second seeds with said liquid;
f) delivering said photoradiation to said first seed comprising converging said photoradiation towards said first seed; and
g) delivering said photoradiation to said second seed comprising diverging said photoradiation away from said second seed;
wherein said likelihood of germination of said seed is increased relative to delivering said photoradiation without converging or diverging said photoradiation.
